(19) United States
(12) Patent Application Publication
(10) Pub. No.: US 2025/0204834 A1
(43) Pub. Date: Jun. 26, 2025

(54) SYSTEM AND METHODS FOR VISUALIZATION OF CARDIAC SIGNALS

(71) Applicant: Anumana, Inc., Cambridge, MA (US)

(72) Inventors: Anand Ramani, Fresno, CA (US); Rohit Jain, Danville, CA (US); Yogisha H J, Bengaluru (IN); Sanjeev Shrinivas Nadapurohit, Thane (IN); Karthik K. Bharadwaj, Bengaluru (IN); Leon Ptaszek, Boston, MA (US)

(73) Assignee: Anumana, Inc., Cambridge, MA (US)

(21) Appl. No.: 18/973,044

(22) Filed: Dec. 8, 2024

Related U.S. Application Data
(60) Provisional application No. 63/614,872, filed on Dec. 26, 2023.

(51) Int. Cl. A61B 5/346; A61B 5/00; A61B 5/339

(52) U.S. Cl. CPC A61B 5/346; A61B 5/339; A61B 5/7264

(57) ABSTRACT

A system for visualization of cardiac signals including at least a processor configured to receive electrocardiogram (ECG) signal data including at least a cardiac signal, label the ECG signal data as a function of an ECG machine learning model, wherein training the ECG machine learning model includes receiving a plurality of de-identified medical data from a medical database, generating ECG training data as a function of the plurality of de-identified medical data, wherein the ECG training data includes the plurality of de-identified medical data correlated to a plurality of signal labels, training the ECG machine learning model as a function of the ECG training data and labeling the ECG signal data as a function of the trained ECG machine learning model, and generate a visualization output as function of the labeled ECG signal data.

### CROSS-REFERENCE TO RELATED APPLICATIONS

This application claims the benefit of priority of U.S. Provisional Patent Application Ser. No. 63/614,872, filed on Dec. 26, 2023, and titled “SYSTEM AND METHOD FOR THE INTELLIGENT DETECTION, LABELING, AND VISUALIZATION OF CARDIAC SIGNALS ENABLED BY MEDICAL CASE DATA ANALYSIS” which is incorporated by reference herein in its entirety.

### FIELD OF THE INVENTION

The present invention generally relates to the field of signal processing. In particular, the present invention is directed to visualization of cardiac signals.

### BACKGROUND

Cardiovascular disease is the most common cause of death worldwide. In recent years, there has been a surge in the burden of cardiovascular disease worldwide. Cardiac arrhythmias are responsible for a large proportion of the morbidity and mortality associated with cardiovascular disease. Ablation procedures have evolved to become an integral part of arrhythmia treatment. Ablation procedures are technically challenging and require that the operator successfully gather clinical data and use this data to make integrative assessments in real time to guide therapy. Current systems used to visualize cardiac signals are inaccurate and cannot be relied upon for use during ablation procedures.

### SUMMARY OF THE DISCLOSURE

In an aspect, a system for visualization of cardiac signals is described. The system includes at least a processor and a memory communicatively connected to the at least a processor. The memory contains instructions configuring the at least a processor to receive electrocardiogram (ECG) signal data including at least a cardiac signal and label the ECG signal data as a function of an ECG machine learning model, wherein training the ECG machine learning model includes, receiving a plurality of de-identified medical data from a medical database, generating ECG training data as a function of the plurality of de-identified medical data, wherein the ECG training data includes the plurality of de-identified medical data correlated to a plurality of signal labels, training the ECG machine learning model as a function of the ECG training data, and labeling the ECG signal data as a function of the trained ECG machine learning model. The processor is further configured to generate a visualization output as function of the labeled ECG signal data and present the visualization output through a graphical user interface.

In another aspect, a method for visualization of cardiac signals is described. The method includes receiving, by at least a processor, electrocardiogram (ECG) signal data including at least a cardiac signal and labeling, by the at least a processor, the ECG signal data as a function of an ECG machine learning model, wherein training the ECG machine learning model includes receiving a plurality of de-identified medical data from a medical database, generating ECG training data as a function of the plurality of de-identified medical data, wherein the ECG training data includes the plurality of de-identified medical data correlated to a plurality of signal labels, training the ECG machine learning model as a function of the ECG training data and labeling the ECG signal data as a function of the trained ECG machine learning model. The method further includes generating, by the at least a processor, a visualization output as function of the labeled ECG signal data and presenting, by the at least a processor, the visualization output through a graphical user interface.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of a system for visualization of cardiac signals;

FIG. 2 illustrates a flowchart for implementation of one or more cardiac analysis phases, in accordance with an example embodiment;

FIG. 3 illustrates a flowchart for implementation of a data validation and standardization process, in accordance with an example embodiment;

FIG. 4 illustrates a flowchart for implementation of a labeling process, in accordance with an example embodiment;

FIG. 5 illustrates a flowchart for implementation of a model development process, in accordance with an example embodiment;

FIG. 6A illustrates a schematic of deep learning architecture for detecting one or more cardiac signals, in accordance with an example embodiment;

FIG. 6B illustrates a block diagram of the deep learning architecture for detecting the one or more cardiac signals, in accordance with an example embodiment;

FIG. 7 illustrates a flowchart for implementation of real-time simulation process, in accordance with an example embodiment;

FIG. 8 illustrates a flowchart for implementation of an expert-correction process, in accordance with an example embodiment;

FIG. 9 illustrates a flowchart for implementation of a semi-supervises pipeline, in accordance with an example embodiment;

FIG. 10 illustrates a block diagram of a system which is used for one or more cardiac analysis, in accordance with an example embodiment of the disclosure;

FIG. 11 is a block diagram of an exemplary machine-learning process;

FIG. 12 is a diagram of an exemplary embodiment of a neural network;

FIG. 13 is a diagram of an exemplary embodiment of a node of a neural network;

FIG. 14 is a flow diagram illustrating an exemplary embodiment of a method for visualization of cardiac signals; and

FIG. 15 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

The present disclosure relates to the field of healthcare and, more specifically, to a software system designed to assist in clinical decision-making for the treatment of cardiac disorders, for example, arrhythmia. The techniques involve the analysis of medical case data to develop personalized strategies for the diagnosis and treatment of arrhythmia.

Atrial fibrillation (AF) is the most common arrhythmia in adults and affects a large number of the adult population. Incidence and prevalence of AF are increasing in association with aging of the population. In certain cases, invasive ablation procedures may be performed as a dominant treatment for atrial fibrillation. Although ablation is more effective than pharmacotherapy, the ablation procedure is not curative. Recent advances in technology, including improvements in the electroanatomic mapping systems and catheters utilized in ablation procedures, have not resulted in consistent improvements in ablation procedural outcomes. Moreover, significant patient morbidity and cost is associated with recurrences of atrial fibrillation after ablation. Improvement in the effectiveness of ablation procedures for atrial fibrillation as well as pre- and post-ablation medical management of atrial fibrillation could produce better patient outcomes and reduce health care costs.

Typically, in order to treat atrial fibrillation, clinicians are required to make multiple integrative assessments of a patient's heart. Given the large volume of data and multiple types of relevant data (surface ECG, intracardiac EGM, cardiac and non-cardiac imaging, patient historical data), clinicians may struggle with timely procurement and processing of large volumes of data for prompt decision and treatment strategy.

Furthermore, ablation of arrhythmias, such as atrial fibrillation, is particularly complex and requires that clinicians make integrative assessments of multiple types of data simultaneously. It is possible that failure of clinicians to detect subtle changes in multiple streams of data contribute to suboptimal effectiveness of atrial fibrillation treatment with the current state of the art. Currently, there are no available clinical decision support tools to assist clinicians in organizing and prioritizing the data that must be analyzed. A clinical decision support tool that collects multiple types of data and draws the clinician's attention to the most relevant findings could improve procedure efficacy and safety.

Existing solutions in the market provide partial automation for data analysis, but they lack the sophistication required to comprehensively assess the diverse factors contributing to cardiac arrhythmias, including atrial fibrillation. Furthermore, the absence of personalized treatment strategies limits the effectiveness of interventions.

There is a recognized need for an innovative software solution that harnesses advanced data analytics, artificial intelligence, and machine learning techniques to enable healthcare professionals to make informed and personalized clinical decisions for patients for the treatment of atrial fibrillation.

Accordingly, an embodiment of the present disclosure discloses a system and a method for detection, labeling, and visualization of the cardiac signals. The system may be configured to detect, label, and visualize the cardiac signals based on medical case data analysis. The system may be configured to standardized de-identified data received from medical institutions, medical equipment, and the like. The system may streamline data collection, validation, and storage, ensuring a conversion of the de-identified data to a canonical format.

The system may specify relevant leads, output directories, and generate test cases for label separation based on configuration settings. The system, by using the labeling tool may label the cardiac signals to overcome challenges associated with the labeling of the cardiac signals.

The system may label the cardiac signal based on signal labels. Further, the signal labels may be generated based on morphology of waveforms at a pixel level. In an example embodiment, the signal labels may be obtained from the clinicians and/or experts. According to some example embodiments, the set of signal labels further comprises at least one of presence of signal label, absence of signal label. According to some example embodiments, the absence of signal label includes one or a combination of a no label, a fusion atrial label, a fusion ventricular label, a degraded electrogram label, a pacing spike label, a noise label, a multiple label, and a future consideration label.

In an example embodiment, the system may be coupled with cloud computing platforms, for example, AWS Cloud S3 to provide efficient transmission and storage of the de-identified data. The integration of the cloud computing platforms with the system may overcome challenges associated with scalability and accessibility.

In an example embodiment, the system may be configured to train the machine learning model based on the medical case data. Further, an offline evaluation may be performed on an output of the machine learning model. In an example embodiment, the system may employ a continuous feedback loop for a real-time simulation, a model tuning, and corrections made by an expert. According to some example embodiments, the system may be further configured to perform a crop operation on electrogram segments to generate one or more samples. In accordance with an embodiment, the system may be further configured to train the ML model based on the generated one or more samples.

In an example embodiment, the system may deploy the machine learning model based on accuracy thresholds and acceptance criteria. The accuracy thresholds and acceptance criteria ensure that the machine learning model satisfies pre-determined accuracy standards, increasing a reliability of the machine learning model.

In an example embodiment, the system may provide a visualization output of the cardiac signals based on multiple types of patient data, including, for example, surface electrocardiogram (ECG), intracardiac electrocardiogram (ECG), cardiac and non-cardiac imaging, and patient historical data. The visualization output may indicate labeled cardiac signals. The visualization may generate the visualization output at multiple points during a patient's care journey, such as before ablation procedure, during ablation procedure, or after ablation procedure. The system is configured to provide the clinician decision support to users, such as health care providers, operators, clinicians, and the like based on the visualization output. To this end, the system may display the visualization output a user interface that may differ in distinct phases of care, such as during deployment of the system through electronic health records (EHR) system, before or after ablation, deployment through electroanatomic mapping system and/or recording system during an ablation procedure.

The foregoing summary is illustrative only and is not intended to be in any way limiting. In addition to the illustrative aspects, embodiments, and features described above, further aspects, embodiments, and features will become apparent by reference to the drawings and the following detailed description. The data utilized by the system described in this document are common to all cardiac arrhythmias. The system utilized in this system is therefore applicable to the treatment of arrhythmias in all cardiac chambers. In addition, this system could be utilized in the targeting of structures adjoining the heart that have an impact of cardiac arrhythmias (including but not restricted to the Ganglionic Plexi adjacent to the posterior aspect of the heart).

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure

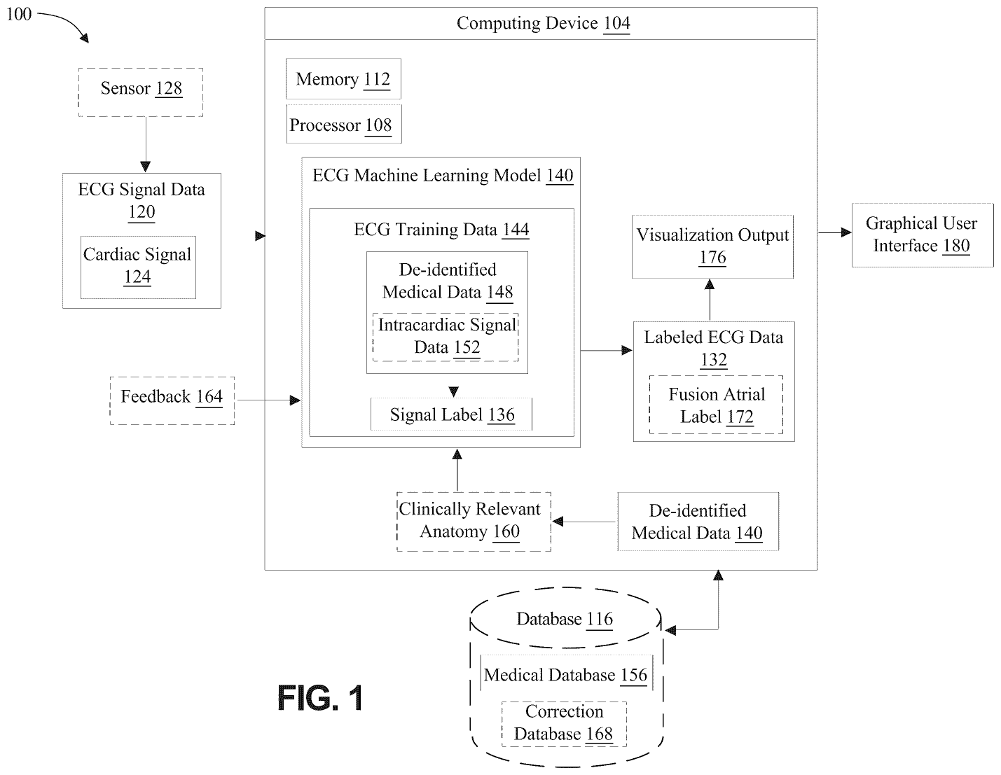

Referring now to FIG. 1, A system for visualization of cardiac signals is described. In one or more embodiments, system 100 may be configured to predict any medical condition and/or medical disease. System 100 includes a computing device 104. System 100 includes a processor 108. Processor 108 may include, without limitation, any processor 108 described in this disclosure. Processor 108 may be included in a and/or consistent with computing device 104. In one or more embodiments, processor 108 may include a multi-core processor. In one or more embodiments, multi-core processor may include multiple processor cores and/or individual processing units. “Processing unit” for the purposes of this disclosure is a device that is capable of executing instructions and performing calculations for a computing device 104. In one or more embodiments, processing units may retrieve instructions from a memory, decode the data, secure functions and transmit the functions back to the memory. In one or more embodiments, processing units may include an arithmetic logic unit (ALU) wherein the ALU is responsible for carrying out arithmetic and logical operations. This may include, addition, subtraction, multiplication, comparing two data, contrasting two data and the like. In one or more embodiments, processing unit may include a control unit wherein the control unit manages execution of instructions such that they are performed in the correct order. In none or more embodiments, processing unit may include registers wherein the registers may be used for temporary storage of data such as inputs fed into the processor and/or outputs executed by the processor. In one or more embodiments, processing unit may include cache memory wherein memory may be retrieved from cache memory for retrieval of data. In one or more embodiments, processing unit may include a clock register wherein the clock register may be configured to synchronize the processor with other computing components. In one or more embodiments, processor 108 may include more than one processing unit having at least one or more arithmetic and logic units (ALUs) with hardware components that may perform arithmetic and logic operations. Processing units may further include registers to hold operands and results, as well as potentially “reservation station” queues of registers, registers to store interim results in multi-cycle operations, and an instruction unit/control circuit (including e.g. a finite state machine and/or multiplexor) that reads op codes from program instruction register banks and/or receives those op codes and enables registers/arithmetic and logic operators to read/output values. In one or more embodiments, processing unit may include a floating-point unit (FPU) wherein the FPU may be configured to handle arithmetic operations with floating point numbers. In one or more embodiments, processor 108 may include a plurality of processing units wherein each processing unit may be configured for a particular task and/or function. In one or more embodiments, each core within multi-core processor may function independently. In one or more embodiments, each core within multi-core processor may perform functions in parallel with other cores. In one or more embodiments, multi-core processor may allow for a dedicated core for each program and/or software running on a computing system. In one or more embodiments, multiple cores may be used for a singular function and/or multiple functions. In one or more embodiments, multi-core processor may allow for a computing system to perform differing functions in parallel. In one or more embodiments, processor 108 may include a plurality of multi-core processors. Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device 104 operating independently or may include two or more computing devices operating in concert, in parallel, sequentially or the like; two or more computing devices may be included together in a single computing device 104 or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device 104. Computing device 104 may include but is not limited to, for example, a computing device 104 or cluster of computing devices in a first location and a second computing device 104 or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device 104, which may operate in parallel, in series, redundantly, or in any other manner used for distribution of tasks or memory 112 between computing devices. Computing device 104 may be implemented, as a non-limiting example, using a “shared nothing” architecture.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 may perform determinations, classification, and/or analysis steps, methods, processes, or the like as described in this disclosure using machine-learning processes. A “machine-learning process,” as used in this disclosure, is a process that automatedly uses a body of data known as “training data” and/or a “training set” (described further below in this disclosure) to generate an algorithm that will be performed by a Processor module to produce outputs given data provided as inputs; this is in contrast to a non-machine learning software program where the commands to be executed are determined in advance by a user and written in a programming language. A machine-learning process may utilize supervised, unsupervised, lazy-learning processes and/or neural networks, described further below.

With continued reference to FIG. 1, system 100 includes a memory 112 communicatively connected to processor 108, wherein the memory 112 contains instructions configuring processor 108 to perform any processing steps as described herein. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a computing device 104. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

With continued reference to FIG. 1, memory 112 may include a primary memory and a secondary memory. “Primary memory” also known as “random access memory” (RAM) for the purposes of this disclosure is a short-term storage device in which information is processed. In one or more embodiments, during use of computing device 104, instructions and/or information may be transmitted to primary memory wherein information may be processed. In one or more embodiments, information may only be populated within primary memory while a particular software is running. In one or more embodiments, information within primary memory is wiped and/or removed after computing device 104 has been turned off and/or use of a software has been terminated. In one or more embodiments, primary memory may be referred to as “Volatile memory” wherein the volatile memory only holds information while data is being used and/or processed. In one or more embodiments, volatile memory may lose information after a loss of power. “Secondary memory” also known as “storage,” “hard disk drive” and the like for the purposes of this disclosure is a long-term storage device in which an operating system and other information is stored. In one or remote embodiments, information may be retrieved from secondary memory and transmitted to primary memory during use. In one or more embodiments, secondary memory may be referred to as non-volatile memory wherein information is preserved even during a loss of power. In one or more embodiments, data within secondary memory cannot be accessed by processor. In one or more embodiments, data is transferred from secondary to primary memory wherein processor 108 may access the information from primary memory.

Still referring to FIG. 1, system 100 may include a database 116. Database may include a remote database 116. Database 116 may be implemented, without limitation, as a relational database, a key-value retrieval database such as a NOSQL database, or any other format or structure for use as database that a person skilled in the art would recognize as suitable upon review of the entirety of this disclosure. Database may alternatively or additionally be implemented using a distributed data storage protocol and/or data structure, such as a distributed hash table or the like. Database 116 may include a plurality of data entries and/or records as described above. Data entries in database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in database may store, retrieve, organize, and/or reflect data and/or records.

With continued reference to FIG. 1, system 100 may include and/or be communicatively connected to a server, such as but not limited to, a remote server, a cloud server, a network server and the like. In one or more embodiments. In one or more embodiments, computing device 104 may be configured to transmit one or more processes to be executed by server. In one or more embodiments, server may contain additional and/or increased processor power wherein one or more processes as described below may be performed by server. For example, and without limitation, one or more processes associated with machine learning may be performed by network server, wherein data is transmitted to server, processed and transmitted back to computing device. In one or more embodiments, server may be configured to perform one or more processes as described below to allow for increased computational power and/or decreased power usage by system computing device 104. In one or more embodiments, computing device 104 may transmit processes to server wherein computing device 104 may conserve power or energy.

With continued reference to FIG. 1, processor 108 is configured to receive Electrocardiogram (ECG) signal data 120. “ECG signal data” as described in this disclosure is information associated with cardiac signals received from an individual. For example, and without limitation, cardiac signal data 120 may include cardiac signals received from one or more leads. In one or more embodiments, cardiac signal data 120 may include a matrix having a plurality of cardiac signals 124 and/or associated time variables. A “matrix” for the purposes of this disclosure is an array of numbers or characters arranged in rows or columns which are used to represent an object or properties of the object. For example, and without limitation, a matrix may be used to describe linear equations, differential equations, in a two-dimensional format. In another non limiting example, a matrix may be used to create graphs based on data points, generate statistical models and the like. In one or more embodiments, matrix and/or ECG signal data 120 may include a plurality of cardiac signals associated with a plurality of time variables. As used in the current disclosure, a “cardiac signal” is a signal representative of electrical activity of a heart. The cardiac signal 124 may consist of several distinct waves and intervals, each representing a different phase of the cardiac cycle. These waves may include the P-wave, QRS complex, T wave, U wave, and the like. The P-wave may represent atrial depolarization (contraction) as the electrical impulse spreads through the atria. The QRS complex may represent ventricular depolarization (contraction) as the electrical impulse spreads through the ventricles. The QRS complex may include three waves: Q wave, R wave, and S wave. The T-wave may represent ventricular repolarization (recovery) as the ventricles prepare for the next contraction. The U-wave may sometimes be present after the T wave, it represents repolarization of the Purkinje fibers. The intervals between these waves may provide information about the duration and regularity of various phases of the cardiac cycle. The cardiac signal 124 may help diagnose various heart conditions, such as arrhythmias, myocardial infarction (heart attack), conduction abnormalities, and electrolyte imbalances. In one or more embodiments, cardiac signals 124 may be received by one or more electrodes connected to the skin of an individual. In one or more embodiments, cardiac signals 124 may represent depolarization and repolarization occurring in the heart. In one or more embodiments, cardiac signals 124 may be captured periodically. For example, and without limitation, every second, every millisecond and the like. In one or more embodiments, each cardiac signal 124 may contain an associated time variable. “Time variable” for the purposes of this disclosure is information indicating the time at which a particular cardiac signal 124 was received. For example, and without limitation, time variable may include, 5 ms, 10 ms, 15 ms and the like. In one or more embodiments, each cardiac signal 124 may contain a time variable. In one or more embodiments, time variable may increase in given increments, such as for example., in increments of 5 ms, wherein a first time variable may include 5 ms and a second time variable may include 10 ms. In one or more embodiments, a combination of a plurality of cardiac signals 124 and correlated time variable may be used to generate a graph illustrating the heart functions of an individual. In one or more embodiments, matrix may include a plurality of cardiac signals 124 and correlated time variable during a given time frame such as, for example, over the span of a second, a minute, an hour, and the like. In one or more embodiments, cardiac signals 124 may be captured as voltages, such as millivolts or microvolts.

With continued reference to FIG. 1, the plurality of cardiac signals may capture a temporal view of cardiac electrical activities. A “temporal view,” as used in the current disclosure, refers to the analysis and visualization of heart-related events and phenomena over time. A temporal view may include patterns, changes, and dynamics of cardiac activity over time. A temporal view may include information surrounding the rhythm of the heart, including the regularity or irregularity of heartbeats. It allows for the identification of various rhythm abnormalities such as tachycardia (fast heart rate), bradycardia (slow heart rate), or arrhythmias (irregular heart rhythms). A temporal view of cardiac activities in three dimensions may refer to a visualization that represents the temporal evolution of cardiac events or phenomena in a three-dimensional space. It provides a comprehensive understanding of how various cardiac activities change over time. The cardiac signal 124 may move through the 3D space of the heart over time. The signal does not just move forward in time, it also moves through the physical space of the heart, from SA node through atria, to AV node, and then through the ventricles. Such movement of the electrical signal through the heart's physical space over time can be referred to as “spatiotemporal excitation and propagation” which could be captured by plurality of cardiac signals 124. It is essentially a way of observing and analyzing the timing and sequence of the heart's electrical activity as it moves through the physical structure of the heart. In the current case the dimensions may include axis representing time, spatial dimensions, and cardiac activity. By combining the temporal, spatial, and cardiac activity dimensions, the temporal view of cardiac activities in three dimensions allows for a comprehensive visualization and analysis of dynamic changes occurring within the heart. It can be used to study phenomena like electrical conduction, ventricular wall motion, valve function, blood flow dynamics, or the interaction between different regions of the heart. This visualization approach provides valuable insights into the complex temporal dynamics of cardiac activities and aids in understanding cardiac function, pathology, and treatment evaluation.

With continued reference to FIG. 1, matrix and/or cardiac signals 124 may be received through one or more input devices. “Input device” for the purposes of this disclosure is a device capable of transmitting information to computing device. For example, and without limitation, input device may include a keyboard, a mouse, a touchscreen, a smartphone, a network server, a sensor 128 and the like. In one or more embodiments, input device may include a sensor 128. In one or more embodiments, matrix and/or cardiac signals 124 may be received by input device and/or sensor 128. As used in this disclosure, a “sensor” is a device that may be configured to detect an input and/or a phenomenon and transmit information related to the detection. Sensor 128 may detect a plurality of data. A plurality of data detected by sensor 128 may include, but is not limited to, cardiac signals, heart rate, blood pressure, electrical signals related to the heart, time variables associated with captured data and the like. In one or more embodiments, and without limitation, sensor 128 may include a plurality of sensors 128. In one or more embodiments, and without limitation, sensor 128 may include one or more electrodes, and the like. Electrodes used for an electrocardiogram (ECG) are small sensors 128 or conductive patches that are placed on specific locations on the body to detect and record the electrical signals generated by the heart. Senor may serve as the interface between the body and the ECG machine, allowing for the measurement and recording of the heart's electrical activity. A plurality of sensors 128 may include 10 electrodes used for a standard 12-lead ECG, placed in specific positions on the chest and limbs of the patient. These electrodes are typically made of a conductive material, such as metal or carbon, and are connected to lead wires that transmit the electrical signals to the ECG machine for recording. In one or more embodiments, plurality of cardiac signals 124 may be associated with a 12-lead electrocardiogram. Proper electrode placement is crucial to ensure accurate signal detection and recording. In one or more embodiments, sensors 128 may include wireless sensors 128 wherein data may be received from sensor 128 to computing device wirelessly. In one or more embodiments, wireless sensors 128 may include Bluetooth enabled ECG sensors, RFID ECG sensors, Wi-Fi enabled ECG sensors and the like. In one or more embodiments, wireless sensors 128 may allow for receipt of data from a distance. In one or more embodiments, wireless sensors 128 may allow for a machine or system to receive data without wires connecting the sensors 128 to computing device. In one or more embodiments, the presence of wires from sensors 128 to computing device may obstruct medical personnel from conducting one or more medical treatment procedures.

With continued reference to FIG. 1, the plurality of sensors 128 may be placed on each limb, wherein there may be at least one sensor on each arm and leg. These sensors may be labeled I, II, III, V1, V2, V3, V4, V5, V6, and the like. For example, Sensor I may be placed on the left arm, Sensor II may be placed on the right arm, and Sensor III may be placed on the left leg. Additionally, a plurality of sensors may be placed on various portions of the patient's torso and chest. For example, a sensor V1 may be placed in the fourth intercostal space at both the right sternal borders and sensor V2 may be fourth intercostal space at both the left sternal borders. A sensor V3 may also be placed between sensors V2 and V4, halfway between their positions. Sensor V4 may be placed in the fifth intercostal space at the midclavicular line. Sensor V5 may be placed horizontally at the same level as sensor V4 but in the anterior axillary line. Sensor V6 may be placed horizontally at the same level as V4 and V5 but in the midaxillary line. In one or more embodiments, each sensor and/or lead may contain a set of electrical signals, wherein matrix may include cardiac signals 124 associated with each lead and/or sensor.

With continued reference to FIG. 1, the plurality of sensors 128 may include augmented unipolar sensors. These sensors may be labeled as aVR, aVL, and aVF. These sensors may be derived from the limb sensors and provide additional information about the heart's electrical activity. These leads are calculated using specific combinations of the limb leads and help assess the electrical vectors in different orientations. For example, aVR may be derived from Sensor II and Sensor III. In another example, aVL may be derived from sensor I and Sensor III. Additionally, aVF may be derived from Lead I and Lead II. The combination of limb sensors, precordial sensors, and augmented unipolar sensors allows for a comprehensive assessment of the heart's electrical activity in three dimensions. These leads capture the electrical signals from different orientations, which are then transformed into transformed coordinates to generate vectorcardiogram (VCG) representing magnitude and direction of electrical vectors during cardiac depolarization and repolarization. Transformed coordinates may include one or more a Cartesian coordinate system (x, y, z), polar coordinate system (r, θ), cylindrical coordinate system (p, y, z), or spherical coordinate system (r, θ, φ). In some cases, transformed coordinates may include an angle, such as with polar coordinates, cylindrical coordinates, and spherical coordinates. In some cases, VCG may be normalized thus permitting full representation with only angles, i.e., angle traversals. In some cases, angle traversals may be advantageously processed with one or more processes, such as those described below and/or spectral analysis.

With continued reference to FIG. 1, in one or more embodiments, sensor 128 may include surface electrodes wherein the surface electrodes may be placed above the skin of a user and used to detect electrical impulses. In one or more embodiments, sensor 128 may further include a wearable ECG monitor wherein the wearable ECG monitor may be wrapped around a limb of the individual and used to detect electrical impulses. In one or more embodiments, sensor 128 may further include a Holter monitor, subdermal needle electrodes, and/or any other sensing device capable of receiving electrical signals.

With continued reference to FIG. 1, matrix may include a plurality of cardiac signals 124 captured at discrete time intervals. In one or more embodiments, matrix may be generated and/or received in a digital imaging and communications in medicine (DICOM) Format, a CSV format, as a spread sheet containing cells for each datum and the like. In one or more embodiments, computing device may receive data in a raw format wherein the data may be converted into a matrix.

With continued reference to FIG. 1, cardiac signals 124 received from each sensor 128 may be referred to as an ‘ECG set.’ In one or more embodiments, an ECG set may include cardiac signals 124 captured from a singular sensor 128 over a given period of time. In one or more embodiments, cardiac signal data 120 may include a plurality of ECG sets wherein each ECG set may correspond to a differing input device differing sensor 128 and the like in contact with an individual. In one or more embodiments, each ECG set may correspond to a different surface electrode in contact with an individual. In one or more embodiments, cardiac signal data 120 may include ECG sets wherein ECG sets include similar timeframes in which cardiac signals 124 are captured. For example, and without limitation, an 8-lead system 100 may include 8 ECG sets wherein each ECG set corresponds to a particular lead.

With continued reference to FIG. 1, processor 108 may be configured to receive cardiac signal data 120 in textual format. A “Textual format” for the purposes of this disclosure is a format in which a set of data is represented by characters, numbers or any other alphanumeric representations. For example, and without limitation, a set of data may be said to be in textual format in instances in which the contents of the file contain only characters of readable material. In one or more embodiments, data in textual format may be contrasted with an image, video and the like. In one or more embodiments, data within a textual format may include machine-readable alphanumeric characters. In one or more embodiments, data within a textual format may include data such as .txt, .docx, .xlsx and the like. In one or more embodiments, cardiac signal data 120 may be received in textual format wherein cardiac signal data 120 may include textual data corresponding to Leads and corresponding voltage signals of the leads.

In one or more embodiments, cardiac signal data 120 may include matrix and/or an array of data wherein matrix may include matrix of size N×T, where N is the number of leads in the ECG and T is the number of voltage signals recorded in that ECG. In one or more embodiments, ‘T’ will depend on the frequency of the acquired cardiac signal data 120 (referred to herein as ‘f’) and the length of the signal in seconds (referred to herein as ‘S’), i.e., T=f*S. In one or more embodiments, matrix may include a two dimensional array having size of N×T wherein N may denote the number and/or particular leads and T may denote the voltage signals. In one or more embodiments, cardiac signal data 120 may be received in a 3-dimensional array of N×T×S wherein s may denote the seconds and/or time corresponding to each voltage signal. In one or more embodiments, cardiac signal data 120 may include a matrix having one or more leads and voltage signals associated with each of the one or more leads. In an embodiment, each lead may be configured to receive voltage signals from a patient wherein cardiac signal data 120 may include voltage signals from each lead on the patient. In one or more embodiments, leads may include any leads as described above. In one or more embodiments, each cardiac signal data 120 may include data received from multiple leads in contact with a patient. In one or more embodiments, processor 108 may be configured to receive cardiac signal data 120 wherein cardiac signal data 120 is associated with a particular individual and/or medical patient. In one or more embodiments, cardiac signal data 120 may contain voltage signals over a given period of time and/or cardiac signals 124. In one or more embodiments, each voltage signal within cardiac signal data 120 may contain corresponding time variable (as described above) wherein time variable denotes the time at which the particular voltage signal was received. In an embodiment, matrix may include an array for each lead wherein the array contains voltage signals and time variables associated with the voltage signals. In one or more embodiments, sensors 128 associated with each lead may be configured to receive voltage signals and corresponding time variables. In one or more embodiments, cardiac signal data 120 may be received from a plurality of patients, from a database 116 116, from a web using a web crawler and the like. In one or more embodiments, each set of cardiac signal data 120 may correspond to a particular individual and/or patient. In one or more embodiments, cardiac signal data 120 may contain cardiac signals 124 received from each sensor 128 of a plurality of sensors 128 that were in contact with a patient. In one or more embodiments, the sensors 128 may be configured to receive cardiac signals 124 and associated time variables denoting the time at which the cardiac signal 124 was received. In one or more embodiments, cardiac signals 124 may be received from an 8 or lead ECG wherein each lead includes a sensor 128 configured to receive cardiac signals 124 from a particular portion of an individual's body. In one or more embodiments, cardiac signal data 120 may contain cardiac signals 124 from multiple electrodes recorded over a similar time frame. For example, and without limitation, cardiac signal data 120 may include cardiac signals 124 received from multiple electrodes over a similar timeframe of 0 to 10 seconds. In one or more embodiments, matrix may include a 2-dimensional array as shown as a non-illustrative example below.

With continued reference to FIG. 1, ECG signal data 120 may include a position datum. A “position datum” for the purposes of this disclosure is information indicating the location on an individual's body, or the device used on an individuals, in which cardiac signal 124 was received. For example, and without limitation, position datum may indicate that the cardiac signal 124 was received from a the right side of a user's heart, from the left side of the use's heart and the like. In one or more embodiments, position datum may further indicate that a particular cardiac signal 124 was received from Lead I, Lead II and the like. In one or more embodiments, position datum may indicate the input device in which cardiac signal 124 was received from. In one or more embodiments, position datum may include a position signal. As used in this disclosure, a “position signal” is a signal generated by a localization system to determine the location of an input device within the body. In one or more embodiments, system 100 may include at least a localization system configured to detect at least a position signal as a function of a location of at least an input device. As used in this disclosure, a “localization system” is a specialized apparatus designed to detect and determine the position of an input device within a body or environment by utilizing position signal. These signals are a function of at least an input device location, enabling precise tracking and navigation during medical procedures. In a non-limiting example, the purpose of at least a localization system is to enhance the safety and efficacy of input device-based interventions by providing critical spatial information.

With continued reference to FIG. 1, at least a localization system may include an electromagnetic localization system. Additionally and or alternatively, at least a localization system may include an ultrasound-based localization system, an optical localization system, and an impedance-based localization system. As used in this disclosure, an “electromagnetic localization system” is a type of localization technology that uses electromagnetic fields to determine the precise position and orientation of objects within a given space. This system typically involves generating a low-frequency electromagnetic field in the area of interest, and then tracking the position of sensors or coils that respond to this field. The sensors may be integrated into input devices or other medical instruments, allowing for accurate real-time tracking of their location and movement within the body. In the context of electroanatomic mapping, the electromagnetic localization system enables the precise localization of at least an input device tip within the heart. This is achieved by placing electromagnetic field generators around the patient and using sensors on at least an input device to detect the field. The system calculates the exact position and orientation of at least an input device by measuring the electromagnetic field's strength and direction at the sensor's location. This information is then transmitted to the processor 108, which uses it to construct a detailed, three-dimensional map of the heart's anatomy. This technology is essential for GUI ding medical procedures such as input device ablation, where precise navigation within the heart is critical. By providing accurate and real-time positional data, the electromagnetic localization system ensures that at least an input device can be maneuvered safely and effectively to target areas of abnormal electrical activity, thereby improving the outcomes of the procedure.

With continued reference to FIG. 1, as used in this disclosure, an “ultrasound-based localization system” is a method used to determine the position and movement of objects within the body by employing high-frequency sound waves. The ultrasound-based localization system may involve the use of an ultrasound transducer that emits sound waves, which then reflect off internal structures and are captured by transducer or other sensors. Continuing, the reflected sound waves are processed to create real-time images or data points that represent the location and motion of the tracked object, such as an input device or other medical instruments. The ultrasound-based localization system may be particularly useful in medical procedures because it provides real-time, non-invasive visualization of internal body structures. The ultrasound-based localization system may allow clinicians to guide instruments accurately within the body, enhancing the precision and safety of procedures like input device ablation, biopsies, or other interventions. This technology is often integrated with other systems to provide comprehensive spatial and functional mapping of the area being treated. For example, at least a localization system may utilize ultrasound technology, where an array of ultrasound transducers is positioned around the patient. At least an input device, may be fitted with miniature ultrasound receivers, detects the emitted ultrasound waves. At least a localization system may calculate at least an input device position based on the time it takes for the ultrasound waves to reach the receivers, allowing for precise localization of at least an input device tip during a procedure.

With continued reference to FIG. 1, as used in this disclosure, “optical localization system” is a method of determining the position and movement of objects using light, typically through the use of cameras and other optical sensors. Optical localization system technology may capture visual data from the tracked object and processes this information to calculate its precise location and trajectory in real-time. In an optical localization system, reflective markers or LED lights may be attached to the object being tracked, such as an input device tip. Cameras positioned around the area capture the light reflected or emitted by these markers, and software algorithms analyze the captured images to triangulate the exact position of the markers. This data is then transmitted to the processor 108, which integrates it with other signals to create a comprehensive map of the object's movement within the heart. This method is highly accurate and provides detailed spatial information, making it particularly useful in medical applications where precise positioning is crucial. Optical localization system can be used in conjunction with other localization methods to enhance the overall accuracy and reliability of the electroanatomic mapping system.

With continued reference to FIG. 1, as used in this disclosure, “impedance-based localization system” is a technique used to determine the position of an input device or other medical device within the body by measuring the electrical impedance between the device and electrodes placed on the patient's body. This method involves passing a small, alternating current through the body and measuring the resulting voltage at different points, allowing the system to calculate the impedance. At least a localization system can then use these impedance measurements to triangulate the exact position of at least an input device tip within the heart or other body cavities. Impedance varies with the distance and the type of tissue between at least an input device and the electrodes, enabling precise tracking of the device's location. This technique is particularly useful in electroanatomic mapping and other procedures requiring accurate real-time positioning of medical instruments within the body. In a non-limiting example, position signal may be generated using electromagnetic fields, ultrasound, or other tracking technologies to provide real-time spatial information about at least an input device position. In a non-limiting example, system 100 may employ other tracking technologies, such as optical localization system or impedance-based localization, to generate position signal. Optical localization system uses cameras and reflective markers on at least an input device to capture its movement and position, while impedance-based localization measures electrical impedance differences between at least an input device and the body tissues. These methods provide accurate real-time spatial information that processor 108 uses alongside potential signal.

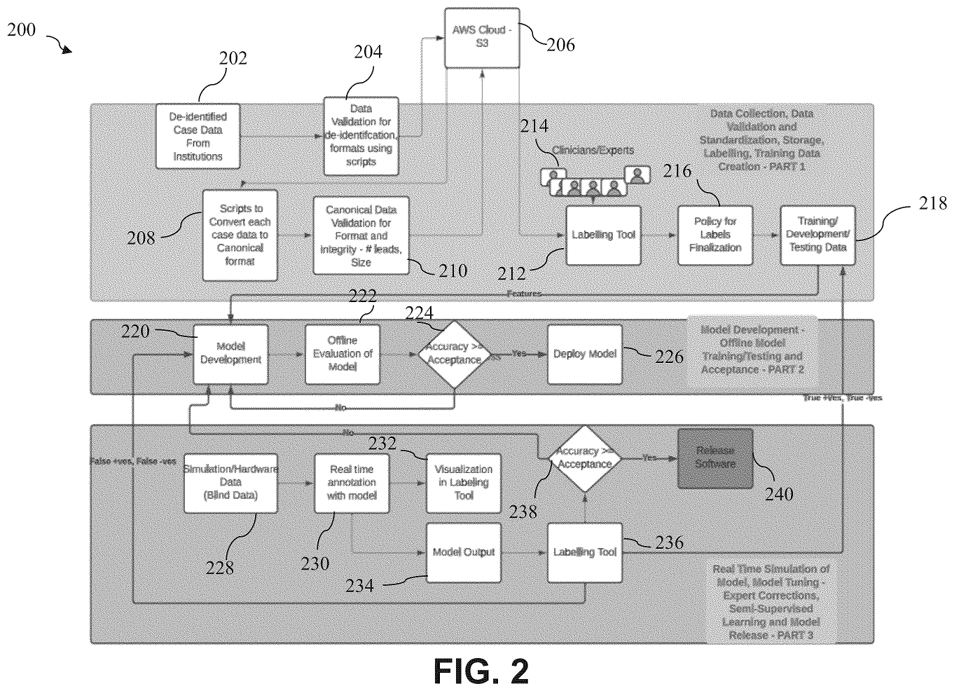

With continued reference to FIG. 1, processor 108 is configured to label ECG signal data 120. In one or more embodiments, a process or labeling ECG signal data 120 may result in the generation of labeled ECG data 132. “Labeled ECG data” or a process of “labeling ECG signal data” for the purposes of this disclosure refers to the detection and labeling of various abnormalities associated with cardiac signals 124. For example, and without limitation, labeled ECG data 132 may indicate that the waveforms of a particular cardiac signal 124 are correlated to individuals with ventricular fusion. In another non limiting example, labeled ECG data 132 may indicate that a particular cardiac signal 124 is weak, cannot be properly read, contains too much noise and the like. In one or more embodiments, labeled ECG data 132 may indicate various heart conditions that an individual may be suffering based on changes and/or abnormalities detected in the cardiac signals 124. In one or more embodiments, processor 108 may label ECG signal data 120 with various labels such as but not limited to, a fusion atrial label 172, a fusion ventricular label (as described in further detail below), a degraded electrogram label, a pacing spike label, a noise label and the like. A “degraded electrogram label” for the purposes of this disclosure refers to information indicating that an electrocardiogram signal has poor quality or has been corrupt in some way. For example, and without limitation, an electrocardiogram signal may be degraded due to electrical noise, poor electrode contact, equipment malfunction, sweating by the patient, the presence of hair between an electrode and the skin and the like. In one or more embodiments, degraded electrocardiogram label may indicate that an electrocardiogram signal and/or cardiac signal may not be suitable for reading. A “pacing spike label” for the purposes of this disclosure is a brief high-amplitude signal seen on an electrocardiogram. In one or more embodiments, the high-amplitude signal may indicate delivery of an electrical impulse used to stimulate the heart muscle in instances in which the heart's natural pacemaker ability is insufficient or absent. In one or more embodiments, pacing spike label may be used to indicate that an electrical impulse was received. A “noise label” for the purposes of this disclosure is information that a particular cardiac signal contains interference. For example, and without limitation, electrical interference may cause noise to occur in a signal. poor electrode contact may cause noise to occur and the like. In one or more embodiments, labeled ECG data 132 may include cardiac signals 124 that have been labeled with identified abnormalities such as but not limited to, abnormalities relating to the recordation of the cardiac signal 124 and/or abnormalities relating to the signal itself which may be indicative of a user's physical health. In one or more embodiments, labeled ECG data 132 may include signal labels 136. A “signal label” for the purposes of this disclosure is information describing an electrocardiogram signal within ECG signal data 120. For example, without limitation signal label 136 may indicate that a cardiac signal 124 is too weak for reading, that a cardiac signal 124 illustrates a particular medical condition and the like. In an embodiment, labeled ECG data 132 may include cardiac signals 124 and correlated signal labels 136, wherein each cardiac signal 124 contains a correlated signal label 136. In one or more embodiments, signal label 136 may contain information indicating a morphology of a waveform within cardiac signal 124. In one or more embodiments labeling ECG signal data 120 may include a process of assigning one or more signal labels 136 to one or more cardiac signals 124 within ECG signal data 120. In one or more embodiments, signal labels 136 may indicate that a cardiac signal 124 is unreadable, a cardiac signal 124 contain a particular morphology and the like. In one or more embodiments, waveforms of cardiac signal 124 may be annotated with signal labels 136 wherein signal labels 136 may include an analysis if the morphology of the waveform. In one or more embodiments, labeling ECG signal data 120 may include any labeling as described in this disclosure such as in reference to at least FIGS. 2-10. In one or more embodiments, cardiac signals 124 may include any cardiac signals 124 as described in this disclosure.

With continued reference to FIG. 1, processor 108 may use a machine learning model to label ECG signal data 120 and/or cardiac signals 124 within ECG signal data 120. In one or more embodiments, computing device 104 may include a machine learning module to implement one or more algorithms or generate one or more machine-learning models to generate outputs. However, the machine learning module is exemplary and may not be necessary to generate one or more machine learning models and perform any machine learning described herein. In one or more embodiments, one or more machine-learning models may be generated using training data. Training data may include inputs and corresponding predetermined outputs so that a machine-learning model may use correlations between the provided exemplary inputs and outputs to develop an algorithm and/or relationship that then allows machine-learning model to determine its own outputs for inputs. Training data may contain correlations that a machine-learning process may use to model relationships between two or more categories of data elements. Exemplary inputs and outputs may come from database 116, user inputs and/or be provided by a user. In other embodiments, a machine-learning module may obtain a training set by querying a communicatively connected database 116 that includes past inputs and outputs. Training data may include inputs from various types of databases 116, resources, libraries, dependencies and/or user inputs and outputs correlated to each of those inputs so that a machine-learning model may determine an output. Correlations may indicate causative and/or predictive links between data, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to categories by tags, tokens, or other data elements. A machine learning module may be used to create a machine learning model and/or any other machine learning model using training data. Training data may be data sets that have already been converted from raw data whether manually, by machine, or any other method. In some cases, the machine learning model may be trained based on user input. For example, a user may indicate that information that has been output is inaccurate wherein the machine learning model may be trained as a function of the user input. In some cases, the machine learning model may allow for improvements to computing device 104 such as but not limited to improvements relating to comparing data items, the ability to sort efficiently, an increase in accuracy of analytical methods and the like.

With continued reference to FIG. 1, in one or more embodiments, a machine-learning module may be generated using training data. Training data may include inputs and corresponding predetermined outputs so that machine-learning module may use the correlations between the provided exemplary inputs and outputs to develop an algorithm and/or relationship that then allows machine-learning module to determine its own outputs for inputs. Training data may contain correlations that a machine-learning process may use to model relationships between two or more categories of data elements. The exemplary inputs and outputs may come from a database 116, and/or be provided by a user. In other embodiments, machine-learning module may obtain a training set by querying a communicatively connected database 116 that includes past inputs and outputs. Training data may include inputs from various types of databases 116, resources, libraries, dependencies and/or user inputs and outputs correlated to each of those inputs so that a machine-learning module may determine an output. Correlations may indicate causative and/or predictive links between data, which may be modeled as relationships, such as mathematical relationships, by machine-learning processes, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. In one or more embodiments, A machine learning model such as ECG machine learning model 140 may include a machine learning model configured to receive inputs such as ECG signal data 120 and/or cardiac signals 124 and output one or more signal labels 136 and/or one or more labeled ECG data 132.

With continued reference to FIG. 1, ECG machine learning model 140 may be configured to receive ECG signal data 120 and/or cardiac signals 124 as an input and output labeled ECG data 132 and/or signal labels 136. In one or more embodiments, ECG training data 144 may include a plurality of ECG signal data 120 and/or a plurality of cardiac signals 124 correlated to a plurality of labeled EG data and/or signal labels 136. In an embodiment, each cardiac signal 124 may contain a correlated signal label 136. In one or more embodiments, ECG training data 144 may be used to train ECG machine learning model 140.

With continued reference to FIG. 1, ECG training data 144 may include de-identified medical data 148. “De-identified medical data” for the purposes of this disclosure refers to health records of multiple individuals in which identifying information has been removed. For example, and without limitation, de-identified medical data 148 may include a medical report of an individual in which the name of the individual, the address of the individual and the like have been removed. In one or more embodiments, de-identified medical data 148 may include any information found within a patient's medical record. For example, and without limitation, de-identified medical data 148 may include lab results, blood test results, previous medication prescribed, previous medical treatment provided, previous cardiac signals 124 recorded from the individual previous diagnosis, such as diagnosis of various medical conditions affecting the individual's health and the like. In one or more embodiments, de-identified medical data 148 may include any information that may be received and/or recorded in the course of receiving medical treatment. In one or more embodiments, de-identified medical data 148 may include medical case data as described in further detail below. In one or more embodiments, de-identified medical data 148 may include medical conditions affecting a user's health such as but not limited to, diabetes, cancer and/or the like. In one or more embodiments, de-identified medical data 148 may include at least intracardiac signal data 152. “Intracardiac signal data” for the purposes of this disclosure refers to electrical signals recorded by electrodes placed within an individual's heart. In one or more embodiments, intracardiac signal data 152 may include intracardiac signals 124 received from one or more sensors as described above. In one or more embodiments, intracardiac signal data 152 may include information received during ablation procedures and/or information received from one or more electrodes placed within a user's heart. In one or more embodiments, processor 108 may be configured to receive a plurality of de-identified medical data 148 wherein each set of de-identified medical data 148 is associated with a singular individual. In one or more embodiments, a plurality of de-identified medical data 148 may be associated with a plurality of individuals. In one or more embodiments, de-identified medical data 148 may be received from a medical database 156. A “medical database” for the purposes of this disclosure is a database 116 that contains medical information. For example, and without limitation, medical database 156 may include a server in which a plurality of electronic health records are stored. In one or more embodiments, database 116 may include medical database 156. In one or more embodiments, medical data may include plurality of de-identified medical data 148 wherein de-identified medical data 148 may include electronic health records. In one or more embodiments, processor 108 may be communicatively connected to medical database 156 wherein processor 108 may be configured to receive a plurality of de-identified medical data 148. In one or more embodiments, de-identified medical data 148 may include any medical data as described in this disclosure. In one or more embodiments, de-identified medical data 148 may include any information used to train one or more machine learning models as described in this disclosure. In one or more embodiments, de-identified medical data 148 may include de-identified data as described in reference to at least FIG. 2.

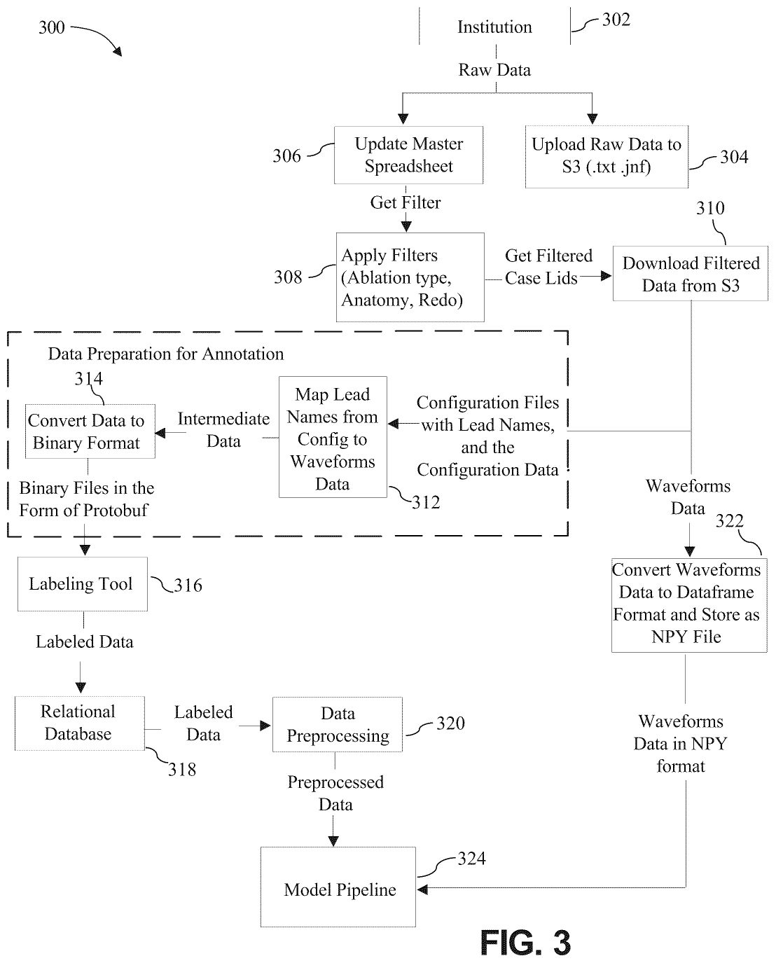

With continued reference to FIG. 1, in one or more embodiments, receiving de-identified medical data 148 may include validating de-identified medical data 148. In one or more embodiments, validating medical data may include validating medical data as a function of a clinically relevant anatomy 160. A “clinically relevant anatomy” for the purposes of this disclosure refers to a body part of an individual for each ECG machine learning model 140 is configured to generate outputs on. For example, and without limitation, ECG machine learning model 140 may be configured to generate outputs only on a left ventricle of the heart wherein the left ventricle may include clinically relevant anatomy 160. In one or more embodiments, in order to increase accuracy of ECG machine learning model 140, ECG machine learning model 140 may be trained to predict outputs only on particular organs. In one or more embodiments, processor 108 may be configured to receive plurality of de-identified medical data 148 containing a plurality of cardiac signals 124. In one or more embodiments, processor 108 may be configured to determine various leads used and/or various portions of the body that have been examined in order to determine if a clinically relevant anatomy 160 exists. In one or more embodiments, processor 108 may utilize a script (such as in reference to at least FIG. 3) in order to validate de-identified medical data 148 and ensure that only de-identified medical data 148 containing clinically relevant anatomies are used. In one or more embodiments, processor 108 may further be configured to filter out de-identified medical data 148 with unreadable cardiac signals 124 and/or cardiac signals 124 that do not contain a sufficient signal length. In one or more embodiments, de-identified medical data 148 may be validated in order to ensure that only medical data useful for property training ECG machine learning model 140 are used. In one or more embodiments, processor 108 may receive plurality of de-identified medical data 148 and generate fileted data, wherein filtered data include de-identified medical data 148 that has been validated. In one or more embodiments, processor 108 may use any process to validate and/or filter de-identified medical data 148 as described in reference to at least FIG. 3. In one or more embodiments, de-identified medical data 148 may undergo one or more cardiac analysis phases as described in reference to at least FIG. 2

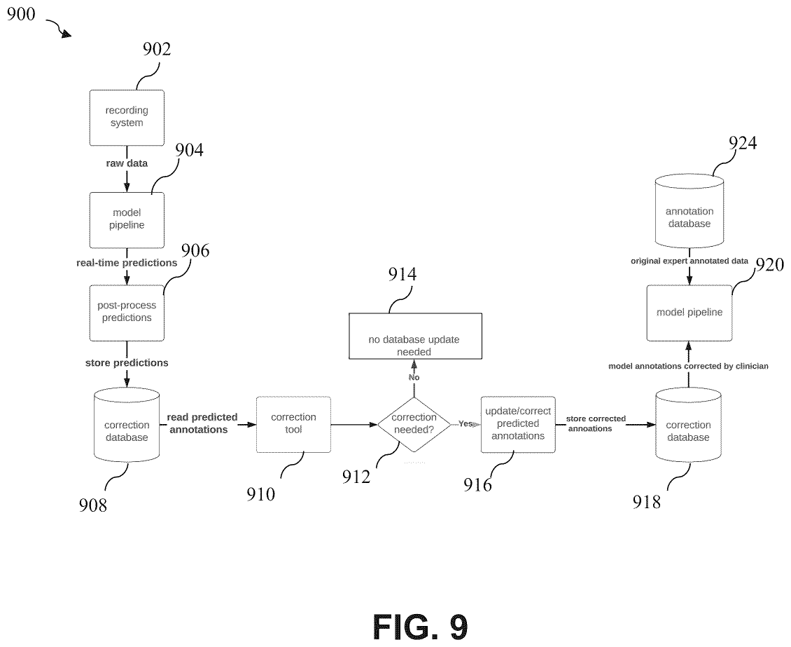

With continued reference to FIG. 1, de-identified medical data 148 may be used to train and/or pretrain ECG machine learning model 140. In one or more embodiments, de-identified medical data 148 and/or cardiac signals 124 within de-identified medical data 148 may be given and/or assigned signal labels 136 wherein de-identified medical data 148 and corresponding signal labels 136 may be used to train ECG machine learning model 140. In one or more embodiments, a medical expert such as for example, a doctor, a physician, a biologist and/or the like may be tasked with assigning signal labels 136 to each set of de-identified medical data 148 of the plurality of medical data. In one or more embodiments, the de-identified medical data 148 and corresponding signal labels 136 may then be used to train ECG machine learning model 140. In one or more embodiments, a portion of plurality of de-identified medical data 148 may be used to pretrain ECG machine learning model 140 wherein another portion may be used as a test set to train ECG machine learning model 140. In an embodiments, ECG training data 144 may include a plurality of de-identified medical data 148 correlated to a plurality of signal labels 136 and/or labeled ECG data 132. In one or more embodiments, an initial set of ECG training data 144 may be created by a medical expert, wherein the initial set may be used to pretrain ECG machine learning model 140. In one or more embodiments, the medical expert may then be tasked with providing feedback 164 to ECG machine learning model 140 in order to provide feedback 164 on predictions made by ECG machine learning model 140. “Feedback” for the purposes of this disclosure refers to an input indicating the accuracy of a prediction made by a machine learning model. For example, and without limitation, feedback 164 may indicate that a signal label 136 generated by ECG machine learning model 140 was accurate wherein ECG machine learning model 140 may be trained to generate more accurate outputs in future iterations. In one or more embodiments, feedback 164 may be received by a user, 3^{rd} party and/or the like. In one or more embodiments, feedback 164 may be received by an input device such as but not limited to any device with a computing system. In one or more embodiments, feedback 164 may include any information that may be used to train one or machine learning models as described in reference to at least FIGS. 2-10. In an embodiment, an initial phase of training ECG machine learning model 140 may include generating ECG training data 144 by assigning signal labels 136 to each set of de-identified medical data 148. In an embodiments, a second phase of training ECG machine learning model 140 may include training ECG machine learning model 140 using ECG training data 144 and a test set. In one or more embodiments, a subsequent phase of training ECG machine learning model 140 may include providing feedback 164 to ECG machine learning model 140 based on predicted outputs. For example, and without limitation, ECG machine learning model 140 may receive cardiac signals 124 and/or a set of ECG medical data and output a predicted signal label 136 and/or labeled ECG data 132. In one or more embodiments, a medical expert may provide feedback 164 on predicted outputs wherein feedback 164 indicating correct and/or incorrect outputs may be used to train ECG machine learning model 140. In one or more embodiments, ECG machine learning model 140 may include a semi supervised machine learning model such as any semi supervised machine learning model as described in this disclosure. In one or more embodiments, processor 108 may implement one or more processes as described in reference to at least FIG. 9, in order to train ECG machine learning model 140 using a semi-supervised process.

In one or more embodiments, a machine learning model such as ECG machine learning model 140 may contain parameter values. “Parameter values” for the purposes of this disclosure are internal variables that a machine learning model has generated from training data in order to make predictions. In one or more embodiments, parameter values may be adjusted during pretraining or training in order to minimize a loss function. In one or more embodiments, during training, predicted outputs of the machine learning model are compared to actual outputs wherein the discrepancy between predicted output and actual outputs are measured in order to minimize a loss function. A loss function also known an “error function” may measure the difference between predicted outputs and actual outputs in order to improve the performance of the machine learning model. A loss function may quantify the error margin between a predicted output and an actual output wherein the error margin may be sought to be minimized during the training process. The loss function may allow for minimization of discrepancies between predicted outputs and actual outputs of the machine learning model. In one or more embodiments, the loss function may adjust parameter values of the machine learning model. In one or more embodiments, in a linear regression model, parameter values may include coefficients assigned to each feature and the bias term. In one or more embodiments, in a neural network, parameter values may include weights and biases associated with the connection between neurons or nodes within layers of the network. In one or more embodiments, during pretraining and/or training of the machine learning model, parameter values of the machine learning model (e.g. ECG machine learning model) may be adjusted as a function of at least one output of the machine learning model. In one or more embodiments, processor 108 may be configured to minimize a loss function by adjusting parameter values of ECG machine learning model 140 based on discrepancies between outputs and feedback 164 associated with said outputs. In one or more embodiments, training ECG machine learning model 140 may include adjusting one or more parameter values of ECG machine learning model 140 based on feedback 164 received. In one or more embodiments, feedback 164 may be used to retrain ECG machine learning model 140. In one or more embodiments, feedback 164 may be used to indicate which inputs and correlated outputs of ECG machine learning model 140 are accurate wherein the inputs and correlated output may be used as training data to retrain ECG machine learning model 140. For example, and without limitation, feedback may indicate that an output of ECG machine learning model such as a particular signal label may be correctly correlated to an input such as ECG signal data. As a result, ECG signal data and correctly correlated signal label may be used to retrain ECG machine learning model. In one or more embodiments, feedback may be used to expand the amount of ECG training data 144 following each iteration of the processing, wherein ECG machine learning model 140 may be configured to generate more accurate outputs in subsequent iterations.

With continued reference to FIG. 1, processor 108 may be configured to generate ECG training data 144 by receiving de-identified medical data 148 and correlated signal labels 136 and/or labeled ECG data 132. In one or more embodiments, a medical expert may label each set of de-identified medical data 148, wherein de-identified medical data 148 and corresponding labels may be used as ECG training data 144. In one or more embodiments, positive feedback 164 to ECG machine learning model 140 may be used as ECG training data 144, wherein for example, feedback 164 indicating that predicted outputs are correct may be used as ECG training data 144 and/or used to train ECG machine learning model 140. In one or more embodiments, ECG machine learning model 140 may be trained as a function of ECG training data 144. In one or more embodiments, processor 108 may then be configured to label ECG signal data 120 as a function of the trained ECG machine learning model 140.

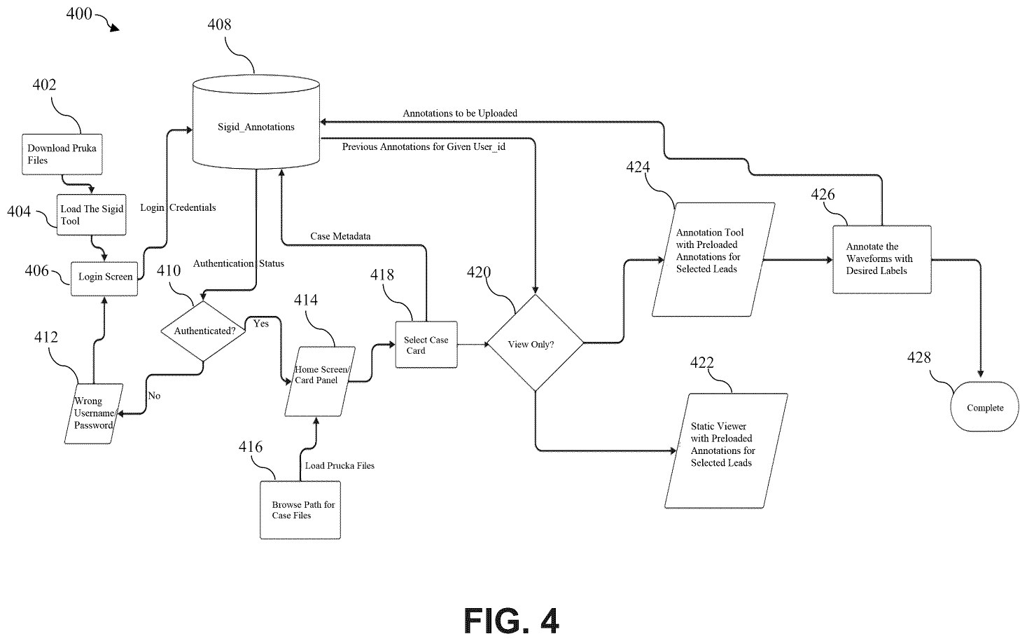

With continued reference to FIG. 1, ECG machine learning model 140 may be iteratively trained with feedback 164. In one or more embodiments, one or more outputs of ECG machine learning model 140 may receive feedback 164 wherein feedback 164 may indicate the accuracy of the output. In one or more embodiments, outputs of ECG machine learning model 140 may be referred to as ‘prediction data’ and/or ‘model prediction data’ as described in reference to at least FIG. 3. In one or more embodiments, model prediction data may be corrected by an expert in the form of feedback 164 in order to train ECG machine learning model 140. In one or more embodiments, outputs of ECG machine learning model 140 may be transmitted to a correction database 168. A “correction database” for the purposes of this disclosure is a database 116 used for recording outputs of ECG machine learning model 140 such that a medical expert may view the outputs and provide feedback 164 to one or more outputs. For example, and without limitation, correction database 168 may contain a plurality of signals labels generated by ECG machine learning model 140 wherein medical expert may provide feedback 164 to each of the one or more outputs. Correction database 168 is described in further detail below such as in reference to at least FIG. 4. In one or more embodiments, training ECG machine learning model 140 may include storing one or more outputs of the ECG machine learning model 140 on correction database 168. In one or more embodiments, training ECG machine learning model 140 may include receiving feedback 164 to the one or more outputs of the machine learning model stored on correction database 168. In one or more embodiments, processor 108 may be configured to store feedback 164 received by medical expert on correction database 168. In one or more embodiments, outputs of ECG machine learning model 140 and correlated feedback 164 may be stored on correction database 168 and used to train ECG machine learning model 140. In one or more embodiments, feedback 164 may be used to modify one or more predicted outputs of ECG machine learning model 140. In one or more embodiments, predicted outputs of ECG machine learning model 140 may include predicted signal labels 136 and/or predicted Labeled ECG data 132. In one or more embodiments, feedback 164 may be used to train ECG machine learning model 140 wherein predicted outputs of ECG machine learning model 140 may change. In one or more embodiments, a process of modifying one or more predicted outputs of ECG machine learning model 140 may include a process of training ECG machine learning model 140 to generate more accurate outputs.

With continued reference to FIG. 1, processor 108 is configured to generate signal labels 136 for ECG signal data 120 and/or labeled ECG data 132. In one or more embodiments, labeled ECG data 132 may include at least a fusion atrial label 172. A “fusion atrial label” for the purposes of this disclosure is a label indicating that electrical impulses from different sources are simultaneously affecting the same area of the heart's atrial chambers. In one or more embodiments, processor 108 may receive cardiac signals 124 and/or ECG signal data 120 and label the inputs with a fusion atrial label 172 indicating that the individual's ECG signals indicate an imbalance in electrical activity of the heart. In one or more embodiments, multiple electrical impulses may act upon the ventricular chambers. In one or more embodiments, labeled ECG data 132 may indicate various portions of ECG signal data 120 that are associated with issues relating to the heart. In one or more embodiments, processor 108 may be configured to label cardiac signals 124 based on identified issues that may be associated with an individual's heart. In one or more embodiments, signal labels 136 may be used to diagnosis an individual with a particular medical condition associated with the heart. For example, and without limitation, signal label 136 may include labels such as not limited to a fusion atrial label 172, a fusion ventricular label, a degraded electrogram label, a pacing spike label, a noise label, a multiple label and the like.

With continued reference to FIG. 1, processor 108 is configured to generate a visualization output 176 as a function of labeled ECG data 132. A “visualization output” for the purposes of this disclosure refers a graphical visualization of cardiac signals 124 and associated signal labels 136. For example, and without limitation, visualization output 176 may include electrocardiogram signals visualized on an X-Y chart with signal labels 136 annotating various abnormalities in the waveforms. A “graphical visualization” for the purposes of this disclosure refers to a visual representation of numerical or alphanumerical data. For example, and without limitation, graphical visualization may include bar charts, line graphs, pie charts, histograms, scatter plots, heat maps, box plots, tree maps, network graphs, two dimensional charts, three dimensional charts and the like. In one or more embodiments, visualization output 176 may include a graphical visualization wherein ECG signal data 120 may be displayed in the form of an X-Y chart. In one or more embodiments, cardiac signals 124 may be plotted on X-Y chart. In one or more embodiments, cardiac signals 124 may contain associated colors based on their signal labels 136. In one or more embodiments, signal labels 136 may be displayed as annotations on the cardiac labels. In one or more embodiments, visualization output 176 may include a two dimension chart showing electrocardiogram signals and/or cardiac signals 124 over a given period wherein the two dimension chart may be annotated with signal labels 136. In one or more embodiments, visualization output 176 may include any graphical format in which ECG signal data 120, labeled ECG data 132, signal labels 136 and the like may be visualized. In one or more embodiments, processor 108 may be configured to generate visualization output 176 such that a user may view cardiac signals 124 in the form of a waveform. In one or more embodiments, visualization output 176 may allow for highlighting and/or annotating of various data of interest, such as for example, data responsible for generation of a particular label. In one or more embodiments, visualization output 176 may include a plurality of cardiac signals 124 within ECG signal data 120 and associated signal labels 136. In one or more embodiments, ECG signal data 120 may include a plurality of cardiac signals 124 over a given period of time. For example, and without limitation, ECG signal data 120 may include cardiac signals 124 recorded prior to an ablation procedure, during an ablation procedure and following an ablation procedure. In one or more embodiments, visualization output 176 may include a comparison between similar cardiac signals 124 before the ablation procedure, during the ablation procure and following the ablation procedure. In one or more embodiments, visualization output 176 may allow a user to visualize changes in cardiac signals 124 over a given period of time and/or changes in signal labels 136. In one or more embodiments, visualization output 176 may include a two-dimensional graphical visualization of one or more cardiac signals 124 within ECG signal data 120 and one or more signals labels for each of the one or more cardiac signals 124. In one or more embodiments, visualization output 176 may be used to for identification of one or more abnormalities within ECG signal data 120. In an embodiments, abnormalities within cardiac signals 124 may indicate underlying medical conditions that an individual may be suffering from. In one or more embodiments, visualization output 176 may allow for identification of an underlying medical condition by visualizing cardiac signals 124 and visualizing signal labels 136. In one or more embodiments, visualization output 176 may include identification of a pulmonary vein potential. A “pulmonary vein potential,” for the purposes of this disclosure, refers to the electrical activity originating from the pulmonary veins. In one or more embodiments, identification of pulmonary vein potential may allow for medical professionals to identify that a heart is not functioning properly. In one more embodiments, in a healthy heart, electrical impulses that control the heartbeats originate from the Sinoatrial node. However, in some cases, pulmonary veins may generate ectopic electrical activity which can disrupt the normal rhythm of the heart and lead to atrial fibrillation. IN one or more embodiments, identification of pulmonary vein potential may include identification of abnormal electrical activity originating from the pulmonary veins. In one or more embodiments, abnormal electrical activity from the veins may result in atrial fibrillation. In one or more embodiments, identifying a pulmonary vein potential may allow a medical professional to isolate these potentials and provide adequate treatment to a user to prevent atrial fibrillation. In one or more embodiments, signal labels 136 may include pulmonary potential potentials, wherein identification of a pulmonary vein potential may include the labeling of a cardiac signal 124 to indicate pulmonary vein potential. In one or more embodiments, visualization output 176 may include a particular cardiac signal 124 and a particular signal label 136 indicating and/or showing pulmonary potential potentials. In one or more embodiments, identification of pulmonary vein potentials may include the use signal labels 136 to label and/or annotate various segments of cardiac signals 124 that resulted in the labeling of cardiac signal 124 with pulmonary potential potentials.

With continued reference to FIG. 1, visualization output 176 may include an identification of abnormal electrical activity. In one or more embodiments, ECG signal data 120 may include recorded electrical activity of an individual's heart. In one or more embodiments signal labels 136 may be used to identify and/or label portions of ECG signal data 120 and/or portions of cardiac signals 124 containing abnormal electrical activity. For the purposes of this disclosure “abnormal electrical activity” refers to electrical activity that is indicative of an underlying medical condition. For example, and without limitation, abnormal electrical activity may

With continued reference to FIG. 1, processor 108 may be configured to create a user interface data structure as a function of at least visualization output 176. As used in this disclosure, “user interface data structure” is a data structure representing a specialized formatting of data on a computer configured such that the information can be effectively presented for a user interface. User interface data structure may include any information as described in this disclosure, such as but not limited to visualization output 176, ECG signal data 120, signal labels 136 and the like.

With continued reference to FIG. 1, processor 108 may be configured to transmit the user interface data structure to a graphical user interface. Transmitting may include, and without limitation, transmitting using a wired or wireless connection, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. Processor 108 may transmit the data described above to database 116 wherein the data may be accessed from database 116. Processor 108 may further transmit the data above to a device display or another computing device 104.

With continued reference to FIG. 1, system may include a graphical user interface (GUI 180). For the purposes of this disclosure, a “user interface” is a means by which a user and a computer system interact. For example, through the use of input devices and software. In some cases, processor 108 may be configured to modify graphical user interface as a function of at least visualization output 176 and visually present visualization output 176 through GUI 180. A user interface may include graphical user interface, command line interface (CLI), menu-driven user interface, touch user interface, voice user interface (VUI), form-based user interface, any combination thereof and the like. In some embodiments, a user may interact with the user interface using a computing device 104 distinct from and communicatively connected to processor 108. For example, a smart phone, smart tablet, or laptop operated by the user and/or participant. A user interface may include one or more graphical locator and/or cursor facilities allowing a user to interact with graphical models and/or combinations thereof, for instance using a touchscreen, touchpad, mouse, keyboard, and/or other manual data entry device. A “graphical user interface,” as used herein, is a user interface that allows users to interact with electronic devices through visual representations. In some embodiments, GUI 180 may include icons, menus, other visual indicators, or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in graphical user interface. Persons skilled in the art, upon reviewing the entirety of this disclosure, will be aware of various ways in which a graphical user interface and/or elements thereof may be implemented and/or used as described in this disclosure.

With continued reference to FIG. 1, GUI 180 may contain one or more interactive elements. An “interactive element,” for the purposes of this disclosure is an element within a graphical user interface that allows for communication with system by a user. For example, and without limitation, interactive elements may include push buttons wherein selection of a push button, such as for example, by using a mouse, may indicate to system to perform a particular function and display the result through graphical user interface. In one or more embodiments, interactive element may include push buttons on GUI 180, wherein the selection of a particular push button may result in a particular function. In one or more embodiments, interactive elements may include words, phrases, illustrations, and the like to indicate the particular process the user would like system to perform. In one or more embodiments, interaction with interactive elements may result in the display of signal labels 136 and/or information associated with signal labels 136.

With continued reference to FIG. 1, system may further include a display device communicatively connected to at least a processor 108. “Display device,” for the purposes of this disclosure is a device configured to show visual information. In some cases, display device may include a liquid crystal display (LCD), a cathode ray tube (CRT), a plasma display, a light emitting diode (LED) display, and any combinations thereof. Display device may include, but is not limited to, a smartphone, tablet, laptop, monitor, tablet, and the like. Display device may include a separate device that includes a transparent screen configured to display computer generated images and/or information. In some cases, display device may be configured to visually present one or more data through GUI 180 to a user, wherein a user may interact with the data through GUI 180. In some cases, a user may view GUI 180 through display device. In one or more embodiments display device may be located on remote device wherein a user may access recipient profile through remove device.

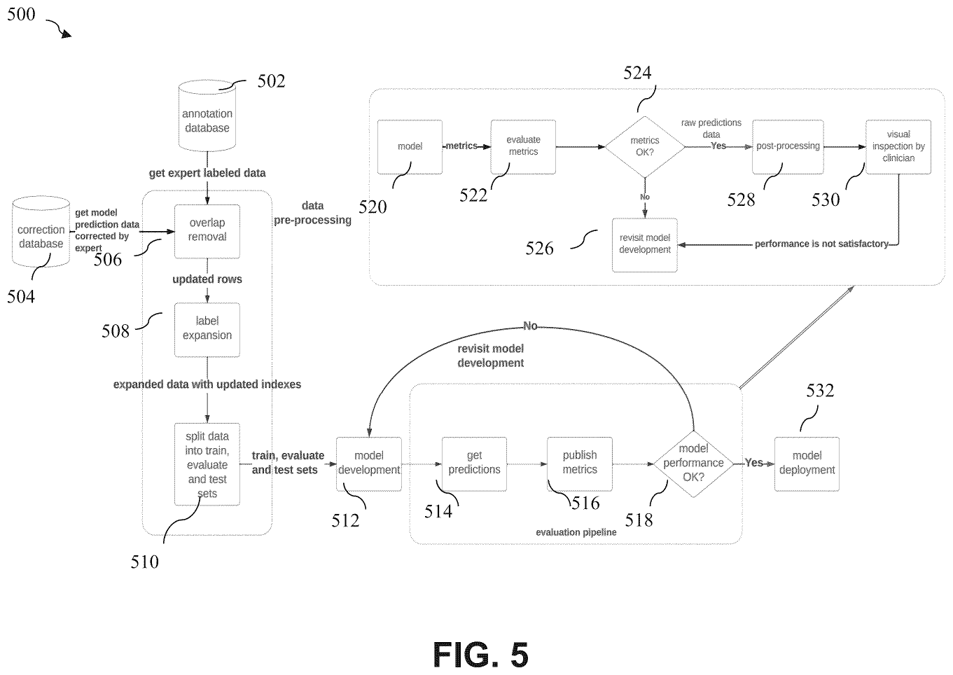

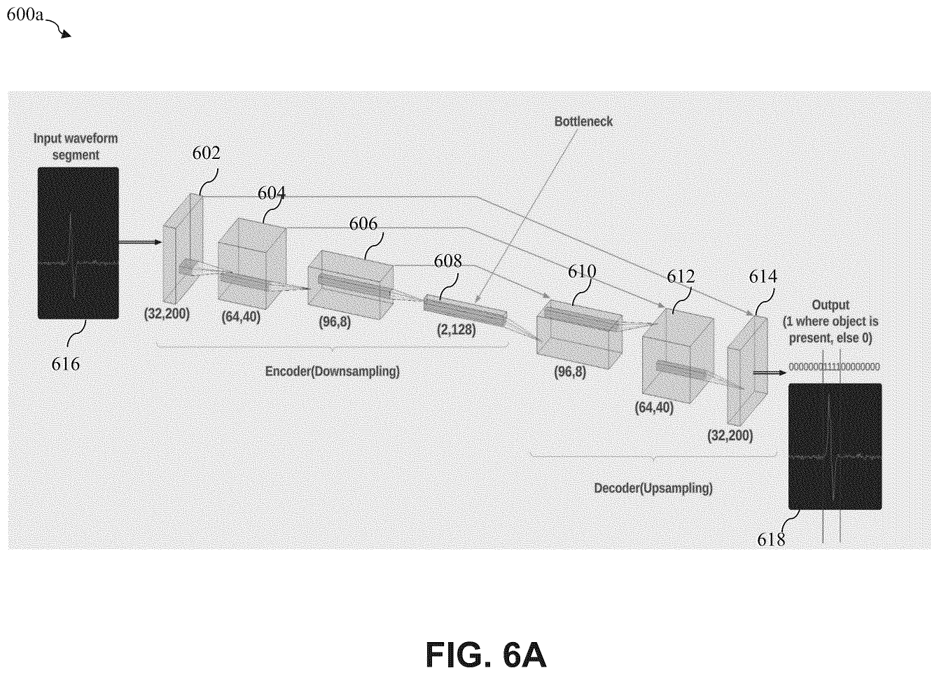

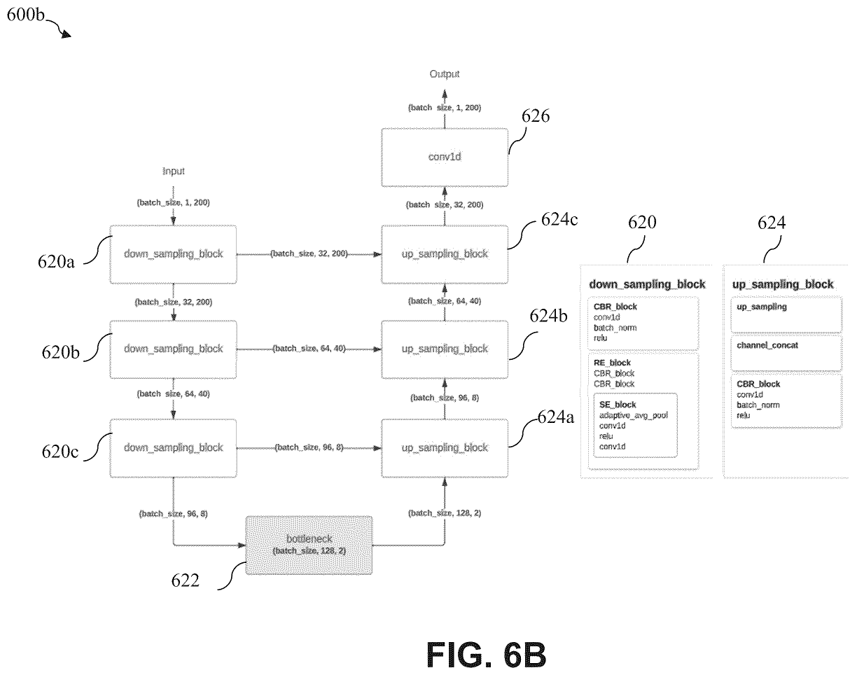

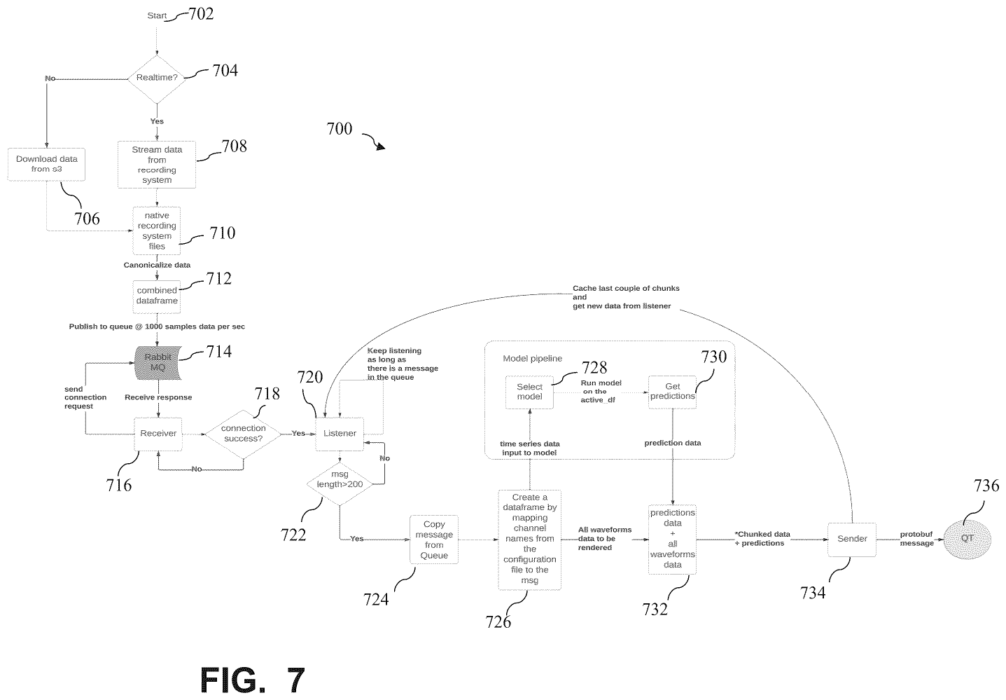

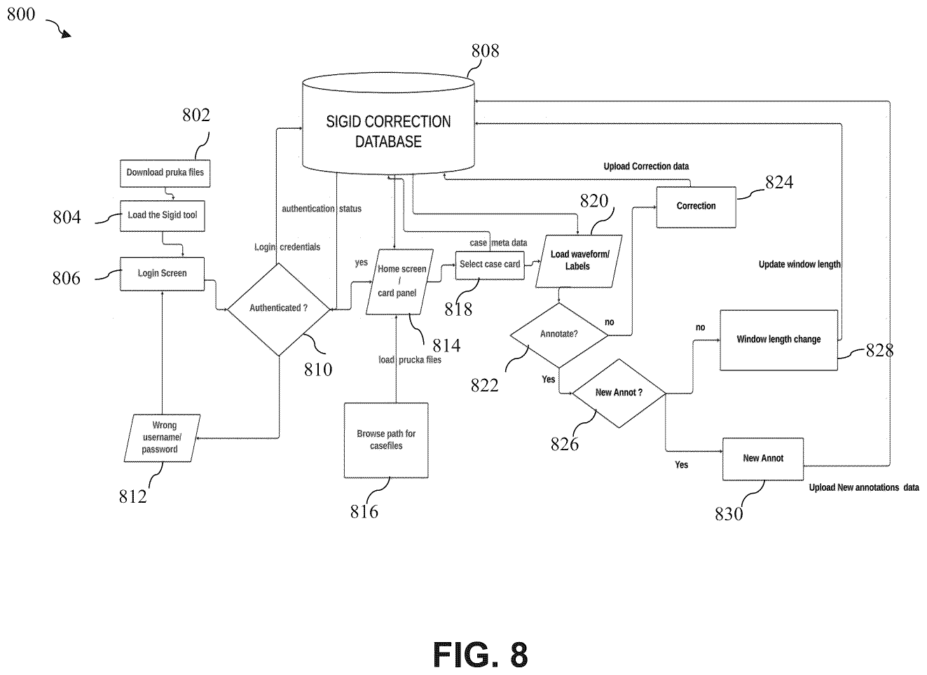

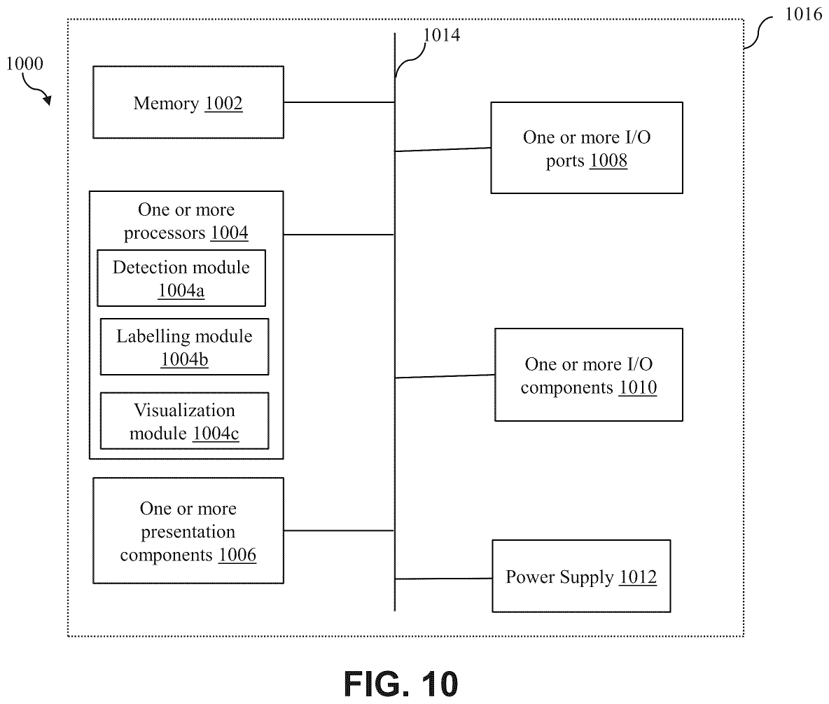

With continued reference to FIG. 1, in one or more embodiments, processor 108 may be configured to receive de-identified medical data 148 and perform one or more cardiac analysis phases as described in reference to at least FIG. 2. In one or more embodiments, processor 108 may be configured to perform data validation on de-identified medical data 148 in any way as described in reference to at least FIG. 3. In one or more embodiments, processor 108 may train ECG machine learning model 140 in any way as described in reference to at least FIG. 4. In one or more embodiments, processor 108 may further train ECG machine learning model 140 in any way as described in reference to at least FIG. 5. In one or more embodiments, system 100 may implement one or more deep learning processes for training of ECG machine learning model 140 as described in reference to at least FIGS. 6A-B. In one or more embodiments, system 100 may implement one or more simulation processes as described in reference to at least FIG. 7. In one or more embodiments, ECG machine learning model 140 may receive feedback 164 similar to any expert-correction process 800 as described in reference to at least FIG. 8. In one or more embodiments, training ECG machine learning model 140 may include any -semi-supervised machine learning processes as described in reference to at least FIG. 9. In one or more embodiments, system 100 may include any system as described in reference to at least FIG. 10.

FIG. 2 illustrates a flowchart for implementation of one or more cardiac analysis phases 200, in accordance with an example embodiment. With reference to FIG. 2, there is shown a one or more cardiac analysis phases 200. The one or more cardiac analysis phases 200 may be performed by a system, or an electronic device such as, but not limited to, a simulation engine, a computing device, a mainframe machine, a server, a computer workstation, a smartphone, a cellular phone, a mobile phone, a gaming device, a consumer electronic (CE) device and/or any other device with simulation capabilities.

The one or more cardiac analysis phases 200 may comprise: (i) a data collection, a data validation, a standardization, a storage labeling, a training data creation—part 1, (ii) a model development—an offline model, a training/testing and an acceptance—part 2, and a real time simulation of model, a model tuning, an expert corrections, a semi-supervised learning and a model release—part 3.

As shown by FIG. 2, at 202, de-identified case data from institutions is received. In some embodiments, the de-identified case data may be referred to as “medical case data”. In an example embodiment, the de-identified case data may include, but are not limited to, intracardiac signal data, echocardiography data, cardiac computed tomography data, cardiac magnetic resonance imaging data, non-cardiac imaging data, historical case data, for example, paroxysmal and persistent AF data, atrial flutter (typical and atypical) data, and historical prior ablation data, and comorbidities data.

At 204, the data validation on the de-identified case data is performed. Additionally, or alternatively, the de-identified data is formatted based on scripts. The scripts may load vital configuration settings from a configuration file. Further, each configuration file name is structured in the following manner: case number comment, where (comment) is comprised of the following structure: rhythm location phase.

The configuration settings play a pivotal role in shaping a data preparation workflow. The configuration settings may include key configuration parameters. The key configuration parameters may include, but are not limited to, a list of leads relevant to the de-identified data, an output directory, a label directory, label data, a list of test cases, a comma separated file (CSV), a list of valid signal lengths. Further, the output directory may be configured to store processed data, a label directory. Further, the signal labels may be separated corresponding to the list of test cases. Further, the signal labels may be generated based on morphology of waveforms at a pixel level. In an example embodiment, the signal labels may be obtained from the clinicians and/or experts. According to some example embodiments, the set of signal labels further comprises at least one of presence of signal label, absence of signal label. According to some example embodiments, the absence of signal label includes one or a combination of a no label, a fusion atrial label, a fusion ventricular label, a degraded electrogram label, a pacing spike label, a noise label, a multiple label, and a future consideration label. The CSV file may include, but is not limited to, metadata associated with the de-identified data. The de-identified data may include, but are not limited to, anatomy data, ablation type data, and the like.

At 206, the validated de-identified data may be stored to a cloud platform, for example, an AWS cloud—S3. In some embodiments, the AWS cloud may be referred to as “S3”. In an example embodiment, the validated de-identified may be stored in a local database.

At 208, each validated de-identified case data is converted to a canonical format based on the scripts. At 210, a converted data validation is performed for formatting and integrity. Additionally, or alternatively, the canonical data validation may be performed on each converted case data based on an identifier associated with leads, a size, and the like. Further, after the canonical data validation, each converted case data may be stored to the cloud platform. In an example embodiment, the converted case data may be stored in the local database. At 212, each converted case data is inputted to a labeling tool. Additionally, or alternatively, the labeling tool may be configured to receive each converted case data from the cloud platform. Further, at 214, clinicians/experts may provide one or more signal labels corresponding to each converted case data. At 216, a policy for the one or more signal labels is generated. At 218, at least one of: training data, development data, and testing data are generated based on the policy and each converted each case data. At 220, a model development process is performed based on a set of features associated with the at least one of: the training data, the development data, and the testing data. At 222, an offline model evaluation is performed. At 224, a determination is made whether a model accuracy is greater than or equal to a model acceptance. If the model accuracy is not greater than or equal to the model acceptance, then a re-iteration of the model development process is performed. If the model accuracy is greater than or equal to the model acceptance, then, at 226, a model deployment is performed. Further, at 228, simulation/hardware data (or blind data) is obtained. At 230, based on the simulation/hardware data, and the blind data, a real time annotation with the model is performed. Further, at 232, a visualization corresponding to the real time annotation may be displayed with the labeling tool. At 234, a model output is generated based on the real time annotation. At 236, the labeling tool may be configured to receive the model output. At 238, a determination is made whether the model accuracy is greater than or equal to the model acceptance. If the model accuracy is not greater than or equal to the model acceptance, then re-iteration of the model development process is performed. However, if the model accuracy is greater than or equal to the model acceptance, at 240, the software is released. Additionally, or alternatively, the at least one of the training data, the development data, and the testing data is updated based on the model output.

FIG. 3 illustrates a flowchart for implementation of a data validation and standardization process 300, in accordance with an example embodiment. At 302, raw data is received from the institutions. At 304, the raw data is uploaded to the S3. The raw data may correspond to a txt format and/or a jnf format. At 306, a master spreadsheet is updated based on the raw data. At 308, filters, for example, an ablation type filter, an anatomy rhythm filter, a redo filter, etc. are obtained and applied on the updated master spreadsheet.

The script may be configured to filter the signal labels based on a specific criteria. For example, the scripts may be configured to filter an invalid anatomy. Further, the signal labels associated with anatomies, for example, a left atrial appendage (LAA) or ‘POST_WALL’ are excluded from a dataset. The exclusion may ensure that only data with clinically relevant anatomies are considered. The scripts may be configured to filter out invalid signal length labels. In an example embodiment, the signal labels with a signal length less than 40 ms for positive categories and less than 150 ms for negative categories may be omitted from the dataset. The omission may ensure that the data is valid and with proper signal labels.

At 310, the filtered data is obtained from the S3. At 312, based on the configuration files with lead names, and the configuration data, the lead names associated with the configuration files are mapped with the waveforms data.

Further, at 314 the waveforms data is converted to intermediate data based on a binary format. At 316, the labeling tool may be configured to receive binary file in a form of protobuf file. At 318, labeled data is stored in a relational database. At 320, a data processing is performed on the labeled data. At 322, the waveforms data is converted to a data frame format and stored in npy file format. At 324, the model pipeline may be configured to receive the preprocessed data, and the waveforms data in the npy file format.

FIG. 4 illustrates a flowchart for implementation of a labeling process 400, in accordance with an example embodiment. At 402, one or more case files, for example, pruka files are obtained. Further, at 404, the labeling tool may be loaded. In an example embodiment, executable instructions for labeling of one or more cardiac signals may be loaded by the system. The one or more cardiac signals may include, but are not limited to, intracardiac signals, surface cardiac signals, and the like. At 406, a user login interface is loaded. In an example embodiment, the user login interface may be displayed. Further, the user login interface may be configured to receive login credentials, for example, a username, a password, and the like. At 408, a database, for example, a signal annotator database may be configured to store the login credentials. Further, at 410, a determination is made whether the received credentials are valid or not. Additionally, or alternatively, the database may be configured to transmit an authentication status for a user authentication. If the user credentials are not valid, then at 412, the user login interface may be displayed iteratively to receive the login credentials. Further, if the login credentials are valid, at 414, a case user interface for selecting one or more case portions is displayed. In some embodiments, the one or more case portions may be referred to as “one or more case panels”. Additionally, at 416, the pruka files may be received by for labeling of the one or more cardiac signals. Further, at 418, each case portion of the one or more case portions may be configured to receive a user input to display the one or more cardiac signals associated with the one or more case portion. Additionally, or alternatively, case meta data associated with the one or more case portions may be stored in the database. Further, at 420, based on previous annotations associated with a user identifier, a determination is made whether a change is required corresponding to the signal labels associated with the one or more cardiac signal or not. If the change is not required, then at 422, a static viewer may be configured to display the one or more cardiac signals with the obtained annotations for selected leads.

In some embodiments, the prediction data may be generated in a form that may be displayed in the visualization tool. Further, ground truth data may be provided by the clinician corresponding to a respective waveform segment. The bin files for the one or more case portions are generated based on a determination that the leads are not associated with any suffix in an end of the bin file indicating the bin file and/or the respective waveform correspond to the ground truth data. Further, the prediction data may indicate a prediction associated with selected segments of the waveforms data corresponding to respective case. The segments of the waveforms may correspond to the same windows that are annotated by the clinicians. Further, the system may be configured to convert the softmax scores to class labels based on the model predictions. Further, during the preprocessing of the training data, a length of the segment may be converted to a value based on an expansion operation. Further, the model may be configured to receive the converted value. Further, a new start index and an end index are generated corresponding to an old start index and an end index. Further, indexes are used as a new start and an end during storing the model prediction to determine the features that the model may extract during prediction. Further, the leads corresponding to the data are stored with suffix_m to indicate the model prediction. Further, an output of a real time streaming or simulation to predict pulmonary vein potentials (PVPs) is stored in the CSVs. In some embodiments, a difference between a segment wise and a real time is in real time whole segments of data is simulated in form of batches of length 200 milliseconds (ms). Further, the model prediction is performed on the batches of length 200 ms.

Further, in a next iteration there is an overlap of the 100 ms data to overcome corner or edge cases. The start index and the end index for each of the segment masked by the model is stored with the leads suffixed with_R indicating the model predictions from the real time annotations. The labeling tool may be configured to store the formats of the data to display the data. Additionally, or alternatively, the real time annotations are stored based on a model name as user that include a unique identifier. Further, in some embodiments, the bin files for the cases to be displayed with the lead, one with suffixed_m and one with suffixed_R. are generated. Further, the cases may be displayed via the labeling tool to compare the model accuracy in a quality of predictions generated by the model in the segment wise and the real time prediction (static view) comparing with the ground truth data. In some cases, at 424, an annotation tools with preloaded annotations for selected leads may be used to annotate various waveforms. However, if there is a need for the change of the signal label, then, at 426, the waveforms are annotated with the signal labels. Additionally, or alternatively, the annotations may be stored in the database. Further, at 428, a user input is received indicative of the completion of the labeling process 400.

FIG. 5 illustrates a flowchart for implementation of a model development process 500, in accordance with an example embodiment. At 502, expert labeled data is obtained for the data processing process from the database, for example, an annotation database. At 504, model prediction data corrected by the expert and or the clinicians is obtained, from the database, for example, a correction database for the data processing process. At 506, an overlapping is removed associated with the expert labeled data, and the model prediction data. At 508, a label expansion is performed. In an example embodiment, the signal labels with signal lengths less than a predetermined threshold (typically 200 ms) may be expanded. The script employs an ‘extend’ factor that is determined based on an absolute difference between the expected signal length and the actual length. Further, the signal labels are updated corresponding to a desired length that ensures a data integrity. Based on a determination that an adjacent signal label may require expansion; the script may adjust the signal label to ensure a continuous signal. Furthermore, based on a determination that the signal overlaps with a preceding label or a succeeding label, the expansion may be performed to maintain signal integrity. In another example embodiment, the expansion may be performed corresponding to negative categories.

In an example embodiment, the script may expand a segment to a right based on a determination that a sum of the extend factor and an end index of a current segment is lesser than a start index of a next segment or a determination that a following segment's label is matched with a current segment's label. Further, in another example embodiment, the script may expand the segment to the left based on a determination that a difference of extend factor and the start index of the current segment is greater than the end index of the previous segment or a determination that the current segment's label and the preceding segment's label are matched.

Further, in another example embodiment, based on a determination that the expansion is not feasible, or signal length is insufficient, useless data is generated. The script may store label statistics, providing insight into the dataset's characteristics. The label statistics may indicate a total number of labels before and after the expansion. Further, the label statistics may assess an impact of the expansion operation. Further, the label statistics may identify the labels with valid signal lengths based on a predetermined criterion. The label statistics may ensure the data quality. Further, the signal labels may be flagged as invalid length based on a determination that the signal labels do not satisfy the defined signal length criteria. Further, the scripts may store a distribution of signal lengths for each signal label that provides a comprehensive view of the dataset.

At 510, the expanded data with updated indexes is split into training dataset, evaluating dataset, and test dataset. The splitting is primarily based on an association with specified test cases to ensure that the expanded data with updated indexes fragmented into the components suitable for model training, evaluation, and testing. Further, a shuffling operation may ensure randomness in an order of data within the training dataset and the test dataset. Further, the shuffling operation may mitigate any potential bias introduced during the data collection process or the labeling process.

Further, a data loader may be configured to load, preprocess, and obtain the data for the model training. The data loader may be configured to perform a negative sample generation and a class-based filtering. Further, the parameters, for example, a positive class (pos class) and negative classes (neg class) are initialized based on the obtained data. The pos class is associated with the yes label, the fusion atrial label, and the like. The neg class is associated with the no label, the fusion ventricular label, the degraded label, the low-amplitude label, the pacing label, the noise label, the future consideration, and the like.

Further, based on a generation of an instance of the neg class, the data loader may be configured to receive a number of arguments update a model behavior, including data, sig len, negative ratio, mode, and the like. The dataset, intended signal length, ratios for the negative samples, mode of operation, and the like are controlled by the parameters. Further, the data loader may be configured to load a list of data points (pid list) from a java script object notation (JSON) file based on the obtained data and the mode. In an example embodiment, the data loader may be configured to processes the loaded pid list. Additionally, or alternatively, the data loader may be configured to apply additional filters based on the parameters, for example, the case, the anatomy, and the like. The data loader may be configured to update the pid list based on the positive classes and the negative classes. Further, the data loader may be configured to determine sizes of the negative samples based on the specified ratio. Further, the data loader may be configured to generate the negative samples based on the determined size of the negative samples.

Further based on a determination that a log attribute is set to a true value, the data loader may display statistics and information about the dataset, including a number of positive samples, a number of negative samples, a number of valid samples, and the like. Further, a weightage calculation (WRS) operation may be performed to overcome an issue of a class imbalance by assigning higher weights to under-represented classes, ensuring a model attention corresponding to the under-represented classes during the model training. Additionally, or alternatively, the higher weights are determined based on an inverse of class frequencies.

Further, a class weight calculation is performed based on a get weights method that determines class weights based on a distribution of classes in the dataset. The weight class calculation may reduce a class imbalance by assigning different weights to different classes. Further, a get item method may allow obtaining individual data points from the dataset. Further, the get item may retrieve signal data associated with a data point, such as a file, a vein, a lead index, start indices, end indices, and the label. Further, the get item may be configured to load the signal data associated with the data point and apply a data augmentation. Further, based on a determination that metadata is set to the true value. Additionally, or alternatively, the get item may generate additional metadata with the signal data and labels.

Further, the scripts may determine a padding corresponding to a specified length (Sig_len), for example, 200 ms. The padding may indicate additional data that may update a signal length corresponding to the specified length. Further, at least two variables, for example, padding_left and padding_right, are initially set to zero. The at least two variables may be used to determine a padding corresponding to a left of the signal and a right side of the signal.

In some embodiments, an amount of padding between a minimum value (min_padding=0) and the remaining padding required is randomly selected based on a training mode that is used to improve the model's performance. The random padding is assigned to a padding_left variable. The randomization may augment the data for the model training by introducing variability. Further, based on a different training mode, the padding_left variable is set to half of the remaining padding to center the signal within the specified length. The centering of the signal reduces biasing.

Further, a portion of the signal is selected between the updated start indices and the end indices, effectively removing or adding padding as needed. The cropped signal is stored in a crop variable. Further, based on a determination that the length of the cropped signal (crop) is greater than the desired sig_len, further adjustments are made. In the training mode, a random start index is selected within a valid range to maintain the signal length equal to sig_len. In other modes, the start index is adjusted to center the signal within the sig_len. However, if the length of the cropped signal (crop) is not equal to sig_len, zero padding is applied to the right side of the signal to make its length match sig_len. If the mask option is enabled, a binary mask is generated. The binary mask may highlight specific regions of the signal. If the data point belongs to the positive class, the region between padding_left and (end_idx−start_idx)+padding_left is set to 1 in the mask. The binary mask is then reshaped into a 1D array and returned with the cropped signal. If the mask option is not enabled, the function returns the processed (cropped) signal without the mask.

At 512, the model is developed based on the training dataset, evaluating dataset, and test dataset. At 514, predictions are obtained by using the model. At 516, metrics are published via the evaluation pipeline. At 518, a determination is made whether the model performance is accurate or not. If the model performance is not accurate, the model is re-developed. Further, at 520, the model may send the metrics for the evaluation. At 522, the metrics are evaluated. At 524, a determination is made whether the metrics are accurate or not. If the metrics are not accurate, then, at 526, the model is re-developed. If the metrics are accurate, then, at 528, a post-processing is performed on the raw predictions data. At 530, a visual inspection by the clinician is performed. Further, if the model performance is accurate, then at 532, the model is deployed.

FIG. 6A illustrates a schematic 600a of an example embodiment of a deep learning architecture for detecting the one or more cardiac signals, in accordance with an example embodiment. The deep learning architecture may include a down-sampling layer 602, a down-sampling layer 604, a down-sampling layer 606, a bottleneck layer 608, an up-sampling layer 610, an up-sampling layer 612, and an up-sampling layer 614. As shown by FIG. 6A, the down-sampling layer 602, the down-sampling layer 604, and the down-sampling layer 606 may be configured to perform decoding on an input waveform segment 616. The bottleneck layer 608 may be configured to perform compression on an output of at least one of the down-sampling layer 602, the down-sampling layer 604, and the down-sampling layer 606. Further, the up-sampling layer 610, the up-sampling layer 612, the up-sampling layer 614 may be configured to perform decoding on an output of the bottleneck layer to generate an output waveform 618 indicative of the detected one or more cardiac signals. The deep learning architecture, using the object detection technique, may allow for the identification of the cardiac disorders. The object detection in real time ensures continuous monitoring of the one or more cardiac signals, aiding in an early detection of the cardiac disorders, for example, arrhythmias. The object detection technique may increase an accuracy of identifying and classifying the one or more cardiac signals.

FIG. 6B illustrates a block diagram 600b of deep learning architecture for detecting the one or more cardiac signals, in accordance with an example embodiment. The block diagram 600b includes a plurality of down sampling blocks 620, for example, a down sampling block 620a, a down sampling block 620b, a down sampling block 620c, a bottleneck block 622, a plurality of up-sampling blocks, for example, an up-sampling block 624a, an up-sampling block 624b, an up-sampling block 624c, and a convolution1d (conv1d) block 626. In some embodiments, the deep learning architecture may correspond to a one dimensional (1D) U-Net model. The 1D U-Net model may be configured to perform semantic segmentation tasks. The plurality of down sampling blocks 620 may correspond to a contracting path that is used for encoding. The plurality of up sampling blocks 624 may correspond to an expanding path that is used for decoding. Further, the U-Net model may include a plurality of skip connections between corresponding layers.

The deep learning architecture may further include a plurality of custom blocks, a convolution-batch-normalization-rectified-linear-unit (CBR) block, a squeeze-excitation block (SE) block, and a residual (RE) block. The CBR block may include a 1D convolution layer, followed by batch normalization, and a rectified linear unit (ReLU) activation function. The CBR block may be configured to apply a convolution operation to an input feature map, normalize an output, and introduce a non-linearity. Further, the SE block may be configured to adaptively recalibrate channel-wise feature responses by explicitly modeling interdependencies between channels. The SE block may be configured to apply a global average pooling to the input tensor that captures global spatial information by reducing spatial dimensions. Further, two 1D convolution layers are used to perform a bottleneck transformation and learn the channel-wise dependencies. Finally, the output of the second convolution is added to the input tensor using an element-wise addition operation. The element-wise addition operation may enable the SE block to adjust the channel-wise feature responses adaptively based on global contextual information. Further, the RE block may include two CBR block layers and a SE block, followed by a concatenation operation that merges the input and the output of the CBR block and the SE block. The RE block may be configured to learn complex features corresponding to an alleviation of a vanishing gradient problem. The residual connection, formed by the addition operation, allows the model to learn both the identity function and complex feature representations. This helps in training deeper networks, and it's beneficial for the overall model performance.

The U-Net model may include down-sampling layers. Further, the down-sampling layers may include CBR block and RE block layers that are responsible for encoding the input signal into feature maps. The down-sampling layers are followed by up-sampling layers that consist of an up-sample operation and CBR block layers. The up-sampling layers are responsible for decoding the feature maps back to the original input size.

The 1D U-Net model may utilize average pooling layers to pool the input features and concatenate the pooled features with the output of the down-sampling layers. The concatenation may allow the deep learning architecture to capture multi-scale information from the input signal. Finally, the 1D convolution layer is used to produce the final output. Further, a sigmoid activation function is applied to the output to convert the predictions into probabilities.

The sigmoid activation function may be configured to squash the model's output between 0 and 1 indicative of the probabilities. The sigmoid activation function may allow overcoming binary classification problems. Specifically, the sigmoid activation function may indicate the model's output as the probability of belonging to a particular class. In some embodiments, during training, the raw output values may be used for computing the loss. Further, based on the application of the sigmoid activation function, during testing, probability-like values for decision-making may be obtained.

Further, the deep learning architecture may use the sigmoid activation function, during testing to obtain the probabilities directly. Further, based on the probabilities and a thresholding, an assessment may be performed, for example, an accuracy, a precision, a recall, a F1-score, and the like. In some embodiments, post-processing steps may be performed during testing to transform the raw outputs to a more interpretable format. For example, each time step may be categorized based on a threshold. Further, based on a minimum number of time steps (predicted point) each segment may be categorized as a single signal label.

Referring back to FIGS. 6A and 6B, the 1D U-Net model may perform the semantic segmentation tasks based on various custom blocks, including the SE block and RE block, to learn the complex features, adaptively recalibrate the channel-wise feature responses, and capture the multi-scale information from the input signal.

In an example embodiment, a first down sampling layer may include a first CBR block, a first RE block, and a second RE block. Further, a second down sampling layer may include a second CBR block, a third RE block, and a fourth RE block. Further, a third down sampling layer may include a third CBR block, and a fifth RE block. Further, a fourth down sampling layer may include a fourth CBR block, and a sixth RE block. Further, a first up-sampling layer may include a first up-sample layer, and a fifth CBR layer. Further, a second up-sampling layer may include a second up-sample layer, and a third up-sampling layer may include a third up-sample layer, and a seventh CBR block. Further, a final output layer may include alD convolution layer.

FIG. 7 illustrates a flowchart for implementation of real-time simulation process 700, in accordance with an example embodiment. As shown by FIG. 7, at 702, the real-time simulation process 700 is started. At 704, a determination is made whether real-time data streaming is feasible or not. If the real-time data streaming is not feasible, then at 706, data is downloaded from the S3. If the real-time streaming is feasible, then at 708, the data is streamed from a recording system. Further, at 710, native recording systems are generated based on the at least one of: the obtained data from the S3 and the obtained stream data from the recording system. At 712, the data is canonicalized to generate a combined data frame. At 714, a message broker, for example, a rabbit MQ is configured to receive the combined data frame. In an example embodiment, the combined data frame is published to a queue with a predetermined speed, for example, 1000 samples data per second. At 716, a receiver, based on a received response, generates a connection request to the Rabbit MQ. At 718, a determination is whether the connection request is established or not. If the connection request is not established, the receiver generates another connection request to the listener. Further, at step 720, the listener may keep listening as long as there is a message in the queue. Further, at 722, a determination is made whether a message length is greater than a predetermined length, for example, 200 bytes. If the message length in not greater than the predetermined length, then the message is sent to the listener. However, if the message length is greater than the predetermined length, then at 724, the message is copied from the queue. At 726, create a data frame by mapping channel names from the configuration file to the message. At 728, time series data is inputted to a selected model. In one or more embodiments, time series data may include any time series data as described in U.S. Nonprovisional application Ser. No. 18/786,066 filed on Jul. 26, 2024, entitled “APPARATUS AND A METHOD FOR A PLURALITY OF TIME SERIES DATA” and having attorney docket no. 1518-093USU1, the entirety of which is incorporated herein by reference. At 730, the selected model may initiate the processing on the active data frame (df) to generate predictions. At 732, based on the prediction data, and each waveform data, chunked data, and the predictions are generated. Further, at 734, the sender may receive the chunked data, and the predictions. At 736, a messaging library, for example, a QT may be configured to receive protobuf message from the sender.

The script may be configured to read data from a file. Further, the script may be configured to transmit the data from the file, using a network connection, for example, a streaming text oriented messaging protocol (STOMP). The script may be configured to define a function send ept(ept_row, conn) that takes a row of data (ept_row) and a connection object (conn) as parameters. This function sends the data to two different destinations (config.ep_destination and config.ecg_egm_queue) using the provided connection. Further, a main function may be configured to initialize a counter (count). Further, the main function may be configured to print a message indicative of the read data. The main function may establish a connection (conn) to the messaging broker based on provided configuration parameters using the stomp library. In an example embodiment, another connection (conn_default) may be established to a default host and a port.

The script may be configured to read the data from a file specified in the configuration (config.ep_data_path). Further, a first row of data is extracted, processed, and transmitted to specified destinations using the send_ept function. In an example embodiment, a loop may be started that continuously sends subsequent rows of data at a rate of approximately one per millisecond. The loop may display a message for every 1000 rows sent. Further, the code may define two functions: ‘pre_processing’ and ‘channel_mapping_extraction’. The two functions may be configured to perform data pre-processing tasks. The two functions may perform pre-processing on the data. The two functions may receive an input including a path to a data file. Further, the two functions may generate an output indicative of a path to a pre-processed data file. The two functions may read the data file as a pandas data frame, assuming that the data is space-separated. The two functions may remove any columns (axis=1) that contain all not a number (NaN) values. The two functions may determine if the pre-processed file already exists with a naming convention that includes “_canonical”. If the file exists, a path to the file may be provided. Otherwise, the two functions may perform the data pre-processing. Further, a path may be constructed for the pre-processed file by appending “_canonical” to the filename part of the input path. Further, based on a determination that the file does not exist, channel mapping information may be extracted from an associated “configuration” file by calling the ‘channel_mapping_extraction’ function. Further, a list may be created, for example, ‘channel_mapping_list’, that is a list of channel numbers (presumably as integers). Further, 1 is subtracted from each element in ‘channel_mappingjlist’ to adjust for a zero-based indexing. Further, the column names of the data frame may be updated based on the adjusted channel numbers. Further, a new data frame may be generated and the values in ‘df’ may be updated based on rounded values of the original data frame. Further, the pre-processed data may be stored as a text file with a constructed filename and an output is generated that is indicative of a path to the pre-processed data file.

In some embodiments, the function may be configured to receive a path of a ‘configuration’ file as input that is associated with the data file. The function may be configured to extract information associated with the channel mapping from the “configuration” file. The function may be configured to determine a line number indicative of a location of text “Channel Number” in the “configuration” file. The line number may correspond to a reference point for extracting channel information. The function may be configured to open the “configuration” file, and read contents associated with the “configuration file”. Further, the function may store the contents in the ‘lines’ list after skipping header rows based on the obtained line number obtained. The function may call another function, ‘find_channel_details’, passing a dataframe created from the ‘lines’ as input. The function may be configured to extract channel details. The channel details may be stored in a list ‘list_of_channels’.

Finally, the function may generate a dictionary called ‘channel_mapping’. Further, keys are channel numbers and values are associated details. Further, the function may provide the ‘channel_mapping’ dictionary. Further, a logger may be configured to log messages to a file (‘app_waveforms.log’). Additionally, or alternatively, ActiveMQ connections are established for sending data and receiving data. Temporary directories may be generated for logs. The script sets up a configuration dictionary (config) to store various settings and parameters. The may subscribe to an ActiveMQ queue to receive cardiac waveform data. The received data is stored in a list called messages (presumably containing timestamps and corresponding values).

The script may be configured to initialize a model based on the specified configuration. The selected model may be loaded, and the script may initiate a loop for continuous data processing. The script may determine if the length of the messages list is greater than a predefined threshold (configurable as config[“ep_samples” ] or 200). The received data may be generated using a utility function. Additional columns may be generated, and the data is structured appropriately. Model prediction is performed using the initialized model, and the result is stored in the prediction data.

Further, a number of data points may be determined to process corresponding to a predetermined number of iterations based on non-overlapping segments. Further, the data is iterated using a sliding window. Further, a non-overlapping portion of the prediction data may be extracted. The overlapping part of the prediction data (e.g., the last config[“overlapping_window” ] data points) for the next iteration may be cached. In some embodiments, an “OR gate” (logical OR operation) may be used to combine predictions from the previous and current iterations. Additionally, the predictions that are cached from the previous iteration may be used, If the predictions are not cached, then, apply the current iteration's predictions. Every lead is treated separately using this reasoning.

If a file for saving predictions already exists, append the new predictions. If the file doesn't exist, create a new file to save the predictions. The predicted data is segmented into subsequences based on certain conditions, and labels are assigned. The labeled data is stored in a pandas dataFrame. A Protocol Buffers message (model_data) is created to package the processed waveform data, labels, and other relevant information.

The packaged data is converted to a base74-encoded string. Based on the configuration (config[“save_ecg_egm_protobuf_zip_fie” ]), the string is either saved to a protobuf file or sent to a specified message queue (config[“output_queue” ]) for further processing or consumption. The script may include error handling mechanisms to manage cases where no waveform data is received. Further, the script may health checks, and if an error counter is reached, then, the script may send a status message and resets the counter. The main loop may be configured to process incoming data in real-time.

FIG. 8 illustrates a flowchart for implementation of an expert-correction process 800, in accordance with an example embodiment. At 802, one or more case files, for example, pruka files are downloaded. Further, at 804, the system may be loaded, for example, the executable instruction for the labeling of the one or more cardiac signals may be loaded by the system. At 806, a user login interface is loaded. Further, the user login interface may be configured to receive login credentials, for example, a username, a password, and the like. At 808, a database may be configured to store the login credentials. Further, at 810, a determination is made whether the received credentials are valid or not. If the user credentials are not valid, then at 812, instructions for the loading of the user login interface are re-loaded. Further, if the login credentials are valid, at 814, a case user interface for selecting one or more case portion is displayed. Additionally, at 816, Pruka files may be received by the system 404 for labeling of the one or more cardiac signals. Further, at 818, each portion of the one or more case portion may receive a user input to display the one or more cardiac signal associated with the one or more case portion. At 820, waveform and labels are loaded. At 822, a determination is made whether the waveform is annotated or not. If the waveform is not annotated, then at 824, correction is performed and the updated correction data is sent to the database, for example, a SigId correction database. If the waveform is annotated, then at 826, new annotation is required or not. If the new annotation is not required, then at 828, an annotation window length is changed, and the updated window length is sent to the SigId correction database. However, if the new annotation is required, then, at 830, the new annotation is added to the waveform. Further, the new annotation data is added to the SigId correction database.

FIG. 9 illustrates a flowchart for implementation of a semi-supervises pipeline 900, in accordance with an example embodiment. At 902, the raw data is obtained by the recording system. At 904, the model pipeline may be configured to generate real-time predictions. Further, at 906, the post-processing is performed on the real-time predictions to generate predictions. At 908, a correction database may be configured to store the generated predictions. At 910, the correction tool may be configured to read the predicted annotations. At 912, a determination is made whether a correction is required or not. If the correction is not required, then, at 914, the correction database is not updated. However, if the correction is required, then, at 916, the predicted annotations are corrected and/or updated. At 918, the correction database may be configured to store the corrected predicted annotations. At 920, the model pipeline may be configured to receive the corrected annotations. Further, at 924, the annotation database may be configured to send original expert annotated data to the model pipeline.

Further, the model may be configured to generate labelled medical signal data for machine learning tasks. The script may perform various data pre-processing steps that allow enhancing the model performance. Further, an EGM signal dataset may include tagged segments. The tagged segments may include, but are not limited to, negative segments (typically 150 ms) and positive segments (typically 50 ms, indicative of pulmonary vein potential (PVP) signal). The model's input size is fixed at 200 ms, which corresponds to 200 data points. Further, the model may generate inputs of length 200 ms segments are randomly cropped around the tagged negative and positive segments. The random cropping may ensure the model to receive diverse samples for the training and the generalization. Further, annotated data with multiple labels from the clinicians are classified into two types, positives samples and negative samples. The positive samples are associated with the yes label, the fusion atrial label. The negative samples are associated with the no label, the fusion ventricular label, the degraded, the low-amplitude label, the pacing spikes, the noise label, a future consideration label.

Further, based on the reception of the labels from the clinicians, the data are extracted from the database corresponding to a desired clinician. Additionally, or alternatively, a date filter may be used to display the data from a particular date. The annotations are mapped to corresponding case ids to obtain a combined-detailed view. The combined-detailed view may indicate overlapping segments of labels and human errors from the clinicians. Further, based on the data quality, the table view of each case in intermediate form may be generated. The table view may include studycase_id that may indicate an annotation time, a start index and an end index of the wave segment. The data is split into multiple chunks, one each per case-id. The model pipeline may be configured to receive the tables and the intermediate form of the raw prucka files. In some embodiments, based on updating definitions of labels, a backup of a corresponding data frame is stored in the S3 with the timestamp and new labels conforming to the setup guidelines are updated to provide data versioning.

In an example, the SigID architecture may be used at different points, such as before ablation, during ablation and post-ablation, of patient care. In these different points or stages of patient care, the SigID architecture may take different types of data for consideration to generate a patient-specific output and a stage or treatment specific output.

As a result, the SigID architecture may provide an ML-based clinical decision support tool for identification of intracardiac electrograms before, during or after ablation procedures. This may help all operators to reduce procedure time and improve results. Different groups of operators may derive distinct advantages from the system. For example, inexperienced/low-volume operators may utilize the system to reduce the likelihood of missing signals during the procedure and thus increase procedural success. Experienced operators may utilize the system to maximize procedural efficiency. The Pulmonary vein potential (PVP) identification during ablation in the context of sinus rhythm or atrial pacing may be performed by the SigID architecture employing object detection model for effective ablation delivery plan and post-procedure management. For example, the SigID architecture may help an operator in understanding if their catheter is in an appropriate location for ablation, in the pre-procedure stage. Moreover, the SigID architecture may help an operator in understanding if the ablation procedure was effective, success rate, and chances of AR recurrence, in the during-procedure stage. In addition, the SigID architecture may help in quick detection of abnormal electrical activity, thereby improving procedural efficiency. Subsequently, the SigID architecture ensures reduced risk of human error which can cause over- or under-ablation, reduced complication risk, better post-procedure success confirmation, and ability to classify/detect other signals of interest during procedure.

FIG. 10 illustrates a block diagram 1000 of a system 1016 of FIG. 1 which is used for one or more cardiac analysis phases 200, in accordance with an example embodiment of the disclosure. The internal components of the system 1016 includes a bus 1014 that directly or indirectly couples the following devices: a memory 1002, one or more processors 1004, one or more presentation components 1006, one or more input/output (I/O) ports 1008, one or more input/output components 1010, and an illustrative power supply 1012. The bus 1014 represents what may be one or more buses (such as an address bus, data bus, or combination thereof). Although the various blocks of FIG. 10 are shown with lines for the sake of clarity, in reality, delineating various components is not so clear, and metaphorically, the lines would more accurately be grey and fuzzy. For example, one may consider a presentation component such as a display device to be an I/O component. It may be understood that the diagram of FIG. 10 is merely illustrative of the system 1016 that can be used in connection with one or more embodiments of the present invention. The distinction is not made between such categories as “user device”, “server”, “computing device”, “laptop,” “hand-held device,” “mobile phone,” “tablet,” etc., as all are contemplated within the scope of FIG. 10.

The memory 1002 includes, but is not limited to, non-transitory computer readable media that stores program code and/or data for longer periods of time such as secondary or persistent long term storage, like RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information. The system 1016 includes one or more processors 1004 that read data from various entities such as the memory 1002 or I/O components 1010. The one or more presentation components 1006 present data indications to the system or a user device. Exemplary presentation components include a display device, speaker, printing component, vibrating component, etc. The one or more I/O ports 1008 allow the system 1016 to be logically coupled to other devices including the one or more I/O components 1010, some of which may be built in. illustrative components include a microphone, joystick, game pad, satellite dish, scanner, printer, wireless device, etc.

Further, the one or more processor 1004 may include one or more processing modules, for example, a detection module 1004a, a labeling module 1004b, and a visualization module 1004c. The detection module 1004a may be configured to detect one or more cardiac signals using a set of machine learning models, for example, the deep learning architecture. The detection module 1004a may employ the object detection technique in real time to ensure continuous monitoring of the one or more cardiac signals, aiding in the early detection of the cardiac disorders, for example, the arrhythmias. The labeling module 1004b may be configured to label one or more segment of the detected one or more cardiac signals by using a set of signal labels. Further, the visualization module 1004c may be configured to display the labeled one or more cardiac signals. Further, the visualization module 1004c may be configured to generate the visualization output to provide the clinical decision support to users before ablation procedure, during ablation procedure, or after ablation procedure. In some embodiments, the detection module 1004a, the labeling module 1004b, and the visualization module 1004c may be referred to as “detection tool”, “labeling tool”, and “visualization tool”, respectively.

Many modifications and other embodiments of the disclosures set forth herein will come to mind to one skilled in the art to which these disclosures pertain having the benefit of the teachings presented in the foregoing descriptions and the associated drawings. It is noteworthy that the system described herein is based on principles common to all cardiac arrhythmias and could therefore be applied to arrhythmias in all cardiac chambers. Therefore, it is to be understood that the disclosures are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

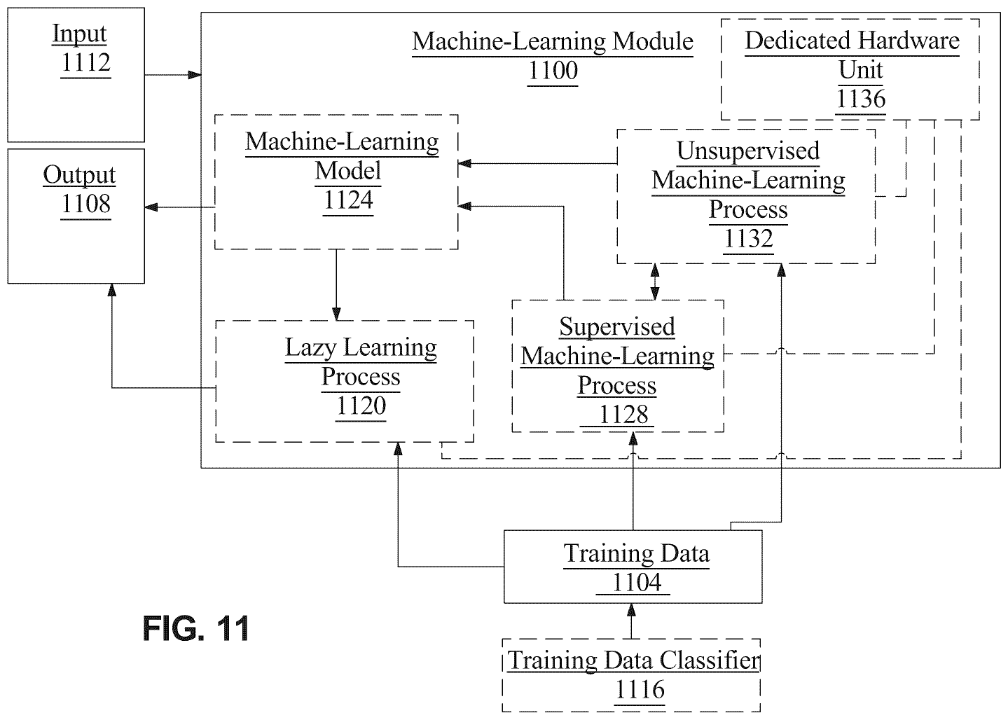

Referring now to FIG. 11, an exemplary embodiment of a machine-learning module 1100 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 1104 to generate an algorithm instantiated in hardware or software logic, data structures, and/or functions that will be performed by a computing device/module to produce outputs 1108 given data provided as inputs 1112; this is in contrast to a non-machine learning software program where the commands to be executed are determined in advance by a user and written in a programming language.

Still referring to FIG. 11, “training data,” as used herein, is data containing correlations that a machine-learning process may use to model relationships between two or more categories of data elements. For instance, and without limitation, training data 1104 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 1104 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 1104 according to various correlations; correlations may indicate causative and/or predictive links between categories of data elements, which may be modeled as relationships such as mathematical relationships by machine-learning processes as described in further detail below. Training data 1104 may be formatted and/or organized by categories of data elements, for instance by associating data elements with one or more descriptors corresponding to categories of data elements. As a non-limiting example, training data 1104 may include data entered in standardized forms by persons or processes, such that entry of a given data element in a given field in a form may be mapped to one or more descriptors of categories. Elements in training data 1104 may be linked to descriptors of categories by tags, tokens, or other data elements; for instance, and without limitation, training data 1104 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 11, training data 1104 may include one or more elements that are not categorized; that is, training data 1104 may not be formatted or contain descriptors for some elements of data. Machine-learning algorithms and/or other processes may sort training data 1104 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 1104 to be made applicable for two or more distinct machine-learning algorithms as described in further detail below. Training data 1104 used by machine-learning module 1100 may correlate any input data as described in this disclosure to any output data as described in this disclosure. As a non-limiting illustrative example inputs may include inputs such as de-identified medical data and outputs may include outputs such as signal labels and/or labeled ECG data.

Further referring to FIG. 11, training data may be filtered, sorted, and/or selected using one or more supervised and/or unsupervised machine-learning processes and/or models as described in further detail below; such models may include without limitation a training data classifier 1116. Training data classifier 1116 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 1100 may generate a classifier using a classification algorithm, defined as a processes whereby a computing device and/or any module and/or component operating thereon derives a classifier from training data 1104. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 1116 may classify elements of training data to various anatomies. For example, and without limitation, training data may be classified to a left ventricle, a right ventricle, to particular lead locations and the like.

Still referring to FIG. 11, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 11, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample—features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 11, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm: l=\sqrt{Σᵢ₌₀ⁿαᵢ²}, where αᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 11, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 11, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 11, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 11, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity, and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 11, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 11, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean upside-effects of compression.

Further referring to FIG. 11, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 11, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation a of a set or subset of values:

$${X}_{n e w} = \frac{X - {X}_{m e a n}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 11, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 11, machine-learning module 1100 may be configured to perform a lazy-learning process 1120 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 1104. Heuristic may include selecting some number of highest-ranking associations and/or training data 1104 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 11, machine-learning processes as described in this disclosure may be used to generate machine-learning models 1124. A “machine-learning model,” as used in this disclosure, is a data structure representing and/or instantiating a mathematical and/or algorithmic representation of a relationship between inputs and outputs, as generated using any machine-learning process including without limitation any process as described above and stored in memory; an input is submitted to a machine-learning model 1124 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 1124 may be generated by creating an artificial neural network, such as a convolutional neural network comprising an input layer of nodes, one or more intermediate layers, and an output layer of nodes. Connections between nodes may be created via the process of “training” the network, in which elements from a training data 1104 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 11, machine-learning algorithms may include at least a supervised machine-learning process 1128. At least a supervised machine-learning process 1128, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include Inputs such as de-identified medical data and/or ECG signal data as described above as inputs, signal labels and/or labeled ECG data as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 1104. Persons skilled in the art, upon reviewing the entirety of this disclosure, will be aware of various possible variations of at least a supervised machine-learning process 1128 that may be used to determine relation between inputs and outputs. Supervised machine-learning processes may include classification algorithms as defined above.

With further reference to FIG. 11, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 11, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 11, machine learning processes may include at least an unsupervised machine-learning processes 1132. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 1132 may not require a response variable; unsupervised processes 1132 may be used to find interesting patterns and/or inferences between variables, to determine a degree of correlation between two or more variables, or the like.

Still referring to FIG. 11, machine-learning module 1100 may be designed and configured to create a machine-learning model 1124 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 11, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 11, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 11, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 11, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 11, one or more processes or algorithms described above may be performed by at least a 11 dedicated hardware unit 1136. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A 11dedicated hardware unit 1136 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such 11dedicated hardware units 1136 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more 11dedicated hardware units 1136 to perform one or more operations described herein, such as evaluation of model and/or algorithm outputs, one-time or iterative updates to parameters, coefficients, weights, and/or biases, and/or any other operations such as vector and/or matrix operations as described in this disclosure.

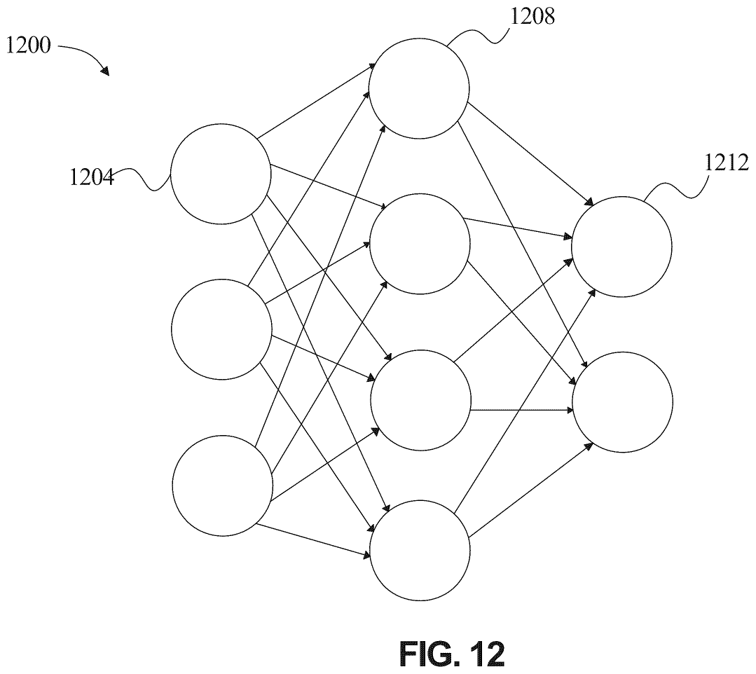

Referring now to FIG. 12, an exemplary embodiment of neural network 1200 is illustrated. A neural network 1200, also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 1204, one or more intermediate layers 1208, and an output layer of nodes 1212. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

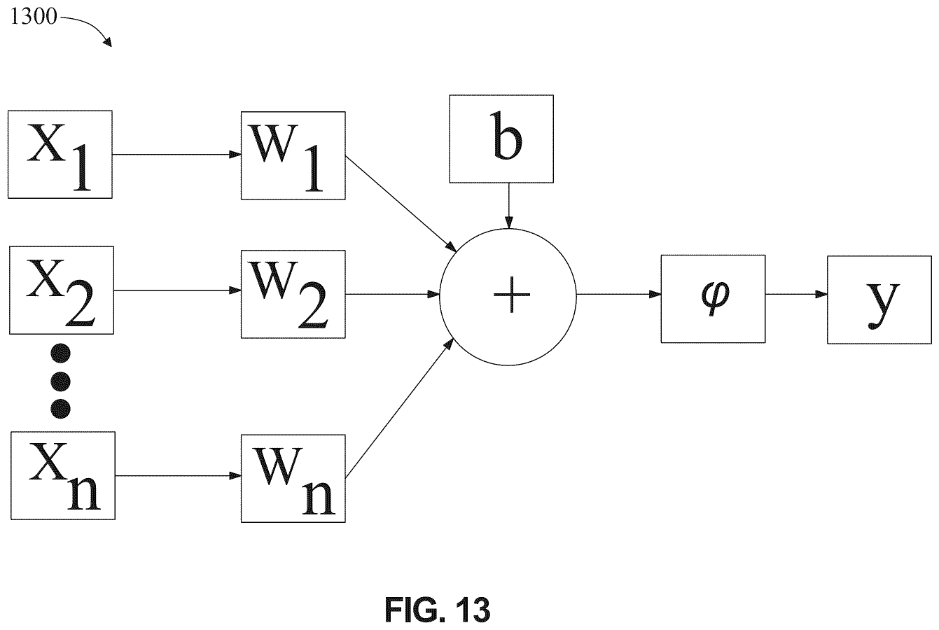

Referring now to FIG. 13, an exemplary embodiment of a node 1300 of a neural network is illustrated. A node may include, without limitation, a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some α, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=α(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

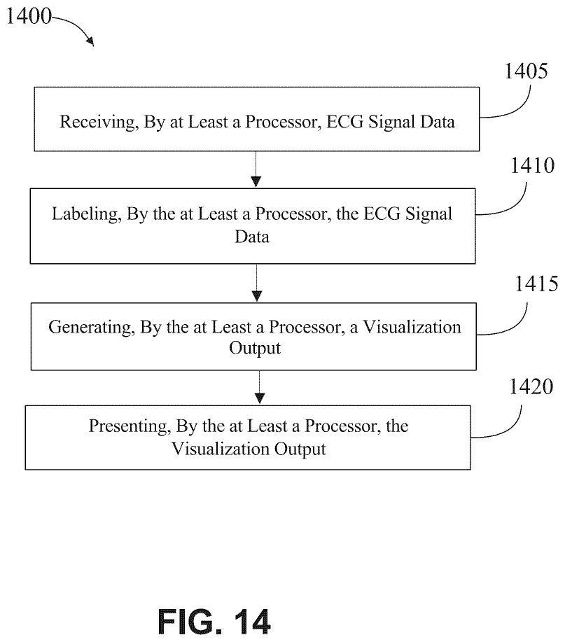

Referring now to FIG. 14, an exemplary method 1400 for visualization of cardiac signals is described. At step 1405, method 1400 includes receiving, by at least a processor, electrocardiogram (ECG) signal data having at least a cardiac signal. This may be implemented with reference to FIGS. 1-13 and without limitation.

With continued reference to FIG. 14, at step 1410, method 1400 includes labeling, by the at least a processor, the ECG signal data as a function of an ECG machine learning model, wherein training the ECG machine learning model includes receiving a plurality of de-identified medical data from a medical database, generating ECG training data as a function of the de-identified medical data, wherein the ECG training data includes the plurality of de-identified medical data correlated to a plurality of signal labels training the ECG machine learning model as a function of the ECG training data and labeling the ECG signal data as a function of the trained ECG machine learning model. In one or more embodiments, training the ECG machine learning model as a function of the ECG training data includes iteratively providing feedback to one or more outputs of the ECG machine learning model. In one or more embodiments, iteratively providing feedback to the one or more outputs of the ECG machine learning model includes storing the one or more outputs of the ECG machine learning model on a correction database, receiving the feedback to the one or more outputs of the machine learning model stored on the correction database, storing the feedback to the one or more outputs on the correction database and modifying one or more predicted outputs of the ECG machine learning model as a function of the feedback and the one or more outputs. In one or more embodiments, receiving the plurality of de-identified medical data from the medical database includes validating the de-identified medical data as a function of at least a clinically relevant anatomy. In one or more embodiments, the ECG machine learning model includes a semi-supervised machine learning model. In one or more embodiments, labeled ECG signal data includes at least a fusion atrial label. In one or more embodiments, de-identified medical data includes at least intracardiac signal data. This may be implemented with reference to FIGS. 1-13 and without limitation.

With continued reference to FIG. 14, at step 1415 method 1400 includes generating, by the at least a processor, a visualization output as function of the labeled ECG signal data. In one or more embodiments, the visualization output includes identification of abnormal electrical activity. In one or more embodiments, visualization output includes identification of a pulmonary vein potential. In one or more embodiments, visualization output includes a two-dimensional graphical visualization of one or more cardiac signals within ECG signal data and one or more signals labels for each of the one or more cardiac signals. This may be implemented with reference to FIGS. 1-13 and without limitation.

With continued reference to FIG. 14, at step 1420 method 1400 includes presenting, by the at least a processor, the visualization output through a graphical user interface. This may be implemented with reference to FIGS. 1-13 and without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

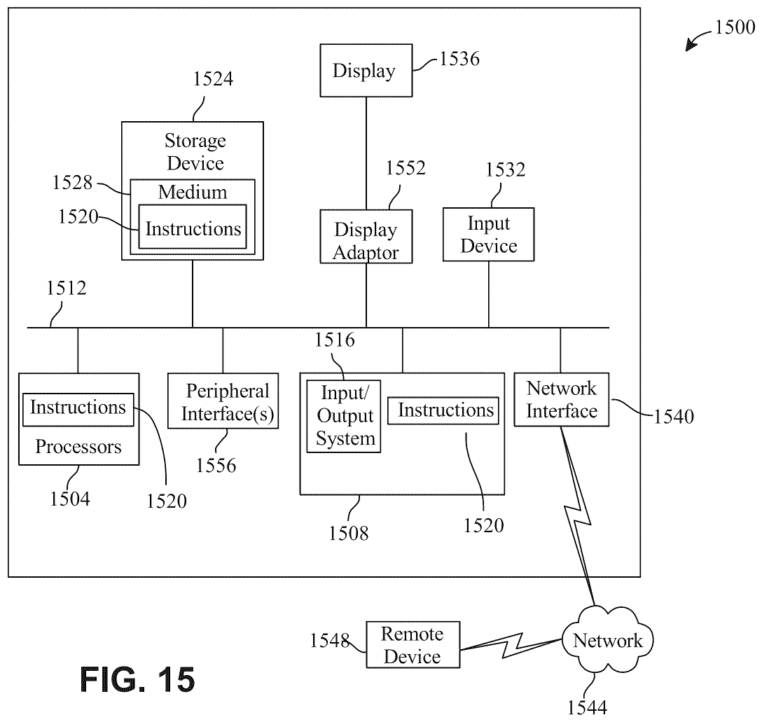

FIG. 15 shows a diagrammatic representation of one embodiment of a computing device in the exemplary form of a computer system 1500 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1500 includes a processor 1504 and a memory 1508 that communicate with each other, and with other components, via a bus 1512. Bus 1512 may include any of several types of bus structures including, but not limited to, a memory bus, a memory controller, a peripheral bus, a local bus, and any combinations thereof, using any of a variety of bus architectures.

Processor 1504 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1504 may be organized according to Von Neumann and/or Harvard architecture as a non-limiting example. Processor 1504 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1508 may include various components (e.g., machine-readable media) including, but not limited to, a random-access memory component, a read only component, and any combinations thereof. In one example, a basic input/output system 1516 (BIOS), including basic routines that help to transfer information between elements within computer system 1500, such as during start-up, may be stored in memory 1508. Memory 1508 may also include (e.g., stored on one or more machine-readable media) instructions (e.g., software) 1520 embodying any one or more of the aspects and/or methodologies of the present disclosure. In another example, memory 1508 may further include any number of program modules including, but not limited to, an operating system, one or more application programs, other program modules, program data, and any combinations thereof.

Computer system 1500 may also include a storage device 1524. Examples of a storage device (e.g., storage device 1524) include, but are not limited to, a hard disk drive, a magnetic disk drive, an optical disc drive in combination with an optical medium, a solid-state memory device, and any combinations thereof. Storage device 1524 may be connected to bus 1512 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1524 (or one or more components thereof) may be removably interfaced with computer system 1500 (e.g., via an external port connector (not shown)). Particularly, storage device 1524 and an associated machine-readable medium 1528 may provide nonvolatile and/or volatile storage of machine-readable instructions, data structures, program modules, and/or other data for computer system 1500. In one example, software 1520 may reside, completely or partially, within machine-readable medium 1528. In another example, software 1520 may reside, completely or partially, within processor 1504.

Computer system 1500 may also include an input device 1532. In one example, a user of computer system 1500 may enter commands and/or other information into computer system 1500 via input device 1532. Examples of an input device 1532 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1532 may be interfaced to bus 1512 via any of a variety of interfaces (not shown) including, but not limited to, a serial interface, a parallel interface, a game port, a USB interface, a FIREWIRE interface, a direct interface to bus 1512, and any combinations thereof. Input device 1532 may include a touch screen interface that may be a part of or separate from display 1536, discussed further below. Input device 1532 may be utilized as a user selection device for selecting one or more graphical representations in a graphical interface as described above.

A user may also input commands and/or other information to computer system 1500 via storage device 1524 (e.g., a removable disk drive, a flash drive, etc.) and/or network interface device 1540. A network interface device, such as network interface device 1540, may be utilized for connecting computer system 1500 to one or more of a variety of networks, such as network 1544, and one or more remote devices 1548 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1544, may employ a wired and/or a wireless mode of communication. In general, any network topology may be used. Information (e.g., data, software 1520, etc.) may be communicated to and/or from computer system 1500 via network interface device 1540.

Computer system 1500 may further include a video display adapter 1552 for communicating a displayable image to a display device, such as display 1536. Examples of a display device include, but are not limited to, a liquid crystal display (LCD), a cathode ray tube (CRT), a plasma display, a light emitting diode (LED) display, and any combinations thereof. Display adapter 1552 and display 1536 may be utilized in combination with processor 1504 to provide graphical representations of aspects of the present disclosure. In addition to a display device, computer system 1500 may include one or more other peripheral output devices including, but not limited to, an audio speaker, a printer, and any combinations thereof. Such peripheral output devices may be connected to bus 1512 via a peripheral interface 1556. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for visualization of cardiac signals, the system comprising:
at least a processor; and
a memory communicatively connected to the at least a processor, the memory containing instructions configuring the at least a processor to:
receive electrocardiogram (ECG) signal data comprising at least a cardiac signal;
label the ECG signal data as a function of an ECG machine learning model,
wherein training the ECG machine learning model comprises:
receiving a plurality of de-identified medical data from a medical database;
generating ECG training data as a function of the plurality of de-identified medical data, wherein the ECG training data comprises the plurality of de-identified medical data correlated to a plurality of signal labels;
training the ECG machine learning model as a function of the ECG training data; and
labeling the ECG signal data as a function of the trained ECG machine learning model;
generate a visualization output as function of the labeled ECG signal data; and
present the visualization output through a graphical user interface.

2. The system of claim 1, wherein the visualization output comprises identification of abnormal electrical activity.

3. The system of claim 1, wherein training the ECG machine learning model as a function of the ECG training data comprises iteratively providing feedback to one or more outputs of the ECG machine learning model.

4. The system of claim 3, wherein iteratively providing feedback to the one or more outputs of the ECG machine learning model comprises:
storing the one or more outputs of the ECG machine learning model on a correction database;
receiving the feedback to the one or more outputs of the machine learning model stored on the correction database;
storing the feedback to the one or more outputs on the correction database; and
modifying one or more predicted outputs of the ECG machine learning model as a function of the feedback and the one or more outputs.

5. The system of claim 1, wherein receiving the plurality of de-identified medical data from the medical database comprises validating the de-identified medical data as a function of at least a clinically relevant anatomy.

6. The system of claim 1, wherein the ECG machine learning model comprises a semi-supervised machine learning model.

7. The system of claim 1, wherein the visualization output comprises an identification of a pulmonary vein potential.

8. The system of claim 1, wherein the plurality of de-identified medical data comprises at least intracardiac signal data.

9. The system of claim 1, wherein the labeled ECG signal data comprises at least a fusion atrial label.

10. The system of claim 1, wherein the visualization output comprises a two-dimensional graphical visualization of one or more cardiac signals within ECG signal data and one or more signals labels for each of the one or more cardiac signals.

11. A method for visualization of cardiac signals, the method comprising:
receiving, by at least a processor, electrocardiogram (ECG) signal data comprising at least a cardiac signal;
labeling, by the at least a processor, the ECG signal data as a function of an ECG machine learning model, wherein training the ECG machine learning model comprises:
receiving a plurality of de-identified medical data from a medical database;
generating ECG training data as a function of the plurality of de-identified medical data, wherein the ECG training data comprises the plurality of de-identified medical data correlated to a plurality of signal labels;
training the ECG machine learning model as a function of the ECG training data; and
labeling the ECG signal data as a function of the trained ECG machine learning model;
generating, by the at least a processor, a visualization output as function of the labeled ECG signal data; and
presenting, by the at least a processor, the visualization output through a graphical user interface.

12. The method of claim 11, wherein the visualization output comprises identification of abnormal electrical activity.

13. The method of claim 11, wherein training the ECG machine learning model as a function of the ECG training data comprises iteratively providing feedback to one or more outputs of the ECG machine learning model.

14. The method of claim 13, wherein iteratively providing feedback to the one or more outputs of the ECG machine learning model comprises:
storing the one or more outputs of the ECG machine learning model on a correction database;
receiving the feedback to the one or more outputs of the machine learning model stored on the correction database;
storing the feedback to the one or more outputs on the correction database; and
modifying one or more predicted outputs of the ECG machine learning model as a function of the feedback and the one or more outputs.

15. The method of claim 11, wherein receiving the plurality of de-identified medical data from the medical database comprises validating the de-identified medical data as a function of at least a clinically relevant anatomy.

16. The method of claim 11, wherein the ECG machine learning model comprises a semi-supervised machine learning model.

17. The method of claim 11, wherein the visualization output comprises an identification of a pulmonary vein potential.

18. The method of claim 11, wherein the plurality of de-identified medical data comprises at least intracardiac signal data.

19. The method of claim 11, wherein the labeled ECG signal data comprises at least a fusion atrial label.

20. The method of claim 11, wherein the visualization output comprises a two-dimensional graphical visualization of one or more cardiac signals within ECG signal data and one or more signals labels for each of the one or more cardiac signals.
